(19) United States
(12) Patent Application Publication
(10) Pub. No.: US 2025/0127787 A1
(43) Pub. Date: Apr. 24, 2025

(54) METHODS FOR TREATING PERIPHERAL NEUROPATHY

(71) Applicant: University of Pittsburgh - Of the Commonwealth System of Higher Education, Pittsburgh, PA (US)

(72) Inventors: Lori Ann Birder, Pittsburgh, PA (US); Edwin Kerry Jackson, Pittsburgh, PA (US); Amanda Sue Wolf-Johnston, Evans City, PA (US)

(73) Assignee: University of Pittsburgh - Of the Commonwealth System of Higher Education, Pittsburgh, PA (US)

(21) Appl. No.: 18/695,777

(22) PCT Filed: Sep. 2, 2022
(86) PCT No.: PCT/US2022/042471

§ 371 Date: Mar. 26, 2024

Related U.S. Application Data
(60) Provisional application No. 63/293,594, filed on Dec. 23, 2021.
(60) Provisional application No. 63/249,487, filed on Sep. 28, 2021.

(51) Int. Cl. A61K 31/522; A61P 25/02

(52) U.S. Cl. CPC A61K 31/522; A61P 25/02

(57) ABSTRACT

Methods for treating a peripheral neuropathy are disclosed herein. Methods are also disclosed for improving nerve function in a subject with a peripheral neuropathy. These methods use a PNPase inhibitor or a PNPase purine nucleoside substrate. These methods include selecting the subject with the peripheral neuropathy; and administering to the subject a therapeutically effective amount of a purine nucleoside phosphorylase (PNPase) inhibitor and/or a PNPase purine nucleoside substrate.

### CROSS REFERENCE TO RELATED APPLICATIONS

This claims the benefit of U.S. Provisional Application No. 63/293,594, filed Dec. 23, 2022, and U.S. Provisional Application No. 63/249,487, filed Sep. 28, 2021, which are both incorporated herein by reference.

### STATEMENT OF GOVERNMENT SUPPORT

This invention was made with Government support under grant number HL109002 awarded by the National Institutes of Health. The government has certain rights in the invention.

### FIELD OF THE DISCLOSURE

This relates to the field of purine nucleoside phosphorylase (PNPase) inhibitors and PNPase purine nucleoside substrates and their use for treating peripheral neuropathy.

### BACKGROUND

Peripheral neuropathy is a condition involving nerve-end damage anywhere in the body. Peripheral neuropathy generally refers to a disorder that affects the peripheral nerves, most often manifested as one or a combination of motor, sensory, sensorimotor, or autonomic neural dysfunction. The morphologies exhibited by peripheral neuropathies can each be attributed to a number of causes. For instance, peripheral neuropathies can be genetically acquired, can result from a systemic disease, can manifest as a post-surgical complication, or can be induced by a toxic agent. Some toxic agents that cause neurotoxicities are therapeutic drugs, antineoplastic agents, contaminants in foods or medicinals, and environmental and industrial pollutants. Generally, treatment involves agents that cause numbness or decrease pain, see for example, U.S. Pat. No. 8,137,711.

Neuropathy also is a major complication of diabetes mellitus. Estimates of the prevalence of neuropathy in diabetes vary widely, from five to eighty percent of patients. In neuropathy of the feet, the loss of sensory information from the foot is related to abnormal and prolonged pressure on the areas of the foot (sensory neuropathy). Motor neuropathy leads to deformity, further increasing pressure loading on the foot. In autonomic neuropathy, loss of innervation of the sweat glands results in dry skin that cracks creating an environment amenable to infection. Autonomic dysfunction contributes further by altering the distribution of micro-circulatory blood flow, directing the blood flow through shunts and away from the nutritive skin capillaries. These factors as a whole, in conjunction with foot trauma, result in skin breakdown and ulcers. The response of the peripheral nervous system to the metabolic effects of diabetes does not appear to differ between type 1 and type 2 diabetes.

A need remains for agents that can be used to treat peripheral neuropathy.

### SUMMARY OF THE DISCLOSURE

Methods for using a PNPase inhibitor or a PNPase purine nucleoside substrate are disclosed herein, such as for treating a peripheral neuropathy. These methods include selecting the subject with the peripheral neuropathy; and administering to the subject a therapeutically effective amount of a purine nucleoside phosphorylase (PNPase) inhibitor and/or a PNPase purine nucleoside substrate, thereby treating the peripheral neuropathy in the subject.

Methods are also disclosed for improving neuron function of a subject with peripheral neuropathy. These methods include selecting the subject with the peripheral neuropathy, wherein the subject is in need of improving function of peripheral nerves, and administering to the subject an amount of a purine nucleoside phosphorylase (PNPase) inhibitor and/or a PNPase purine nucleoside substrate effective to improve function of peripheral neurons in the subject.

The foregoing and other features and advantages of the invention will become more apparent from the following detailed description of several embodiments which proceeds with reference to the accompanying figures.

### BRIEF DESCRIPTION OF THE FIGURES

FIGS. 1A to 1C. Pharmacokinetics of oral 8-aminoguanine (8-AG) administration. FIG. 1A show that in SD rats, the plasma disposition of 8-AG shows a bi-exponential decay, typical of most drugs. The 1^{st} phase of decay represents the distribution of 8-AG from the plasma into the tissues (half-life of 0.38 hours) and the 2^{nd} phase represents elimination from the body (half-life of 37 hours). The data shows that the drug is eliminated slowly enough to be given systemically. In addition, the guanosine to guanine ratio (FIG. 1B) is a good indication that 8-AG blocks PNPase which is sustained and (FIG. 1C) hypoxanthine levels in the urine decrease.

FIGS. 2 and 3. CYP—CIPN (Chemotherapy-induced peripheral neuropathy) alters both superoxide dismutase (SOD1) and glutathione peroxidase activity (GPx) in CIPN rat peripheral (sciatic) nerves. FIG. 2 shows that there was a significant decrease in SOD1 expression in the CIPN rat peripheral nerves versus controls which is normalized by 8-AG treatment. SOD1 is considered the chief defense against activated oxygen free radicals and a decrease in SOD1 can lead to excessive levels of H₂O₂ and a highly toxic and tissue damaging hydroxyl radical. Further, GPx activity in CIPN rat peripheral nerves was significantly elevated which is normalized by 8-AG treatment (FIG. 3). Increases in GPx activity after nerve injury is crucial to protect the nervous tissue against augmented levels of free radicals (i.e., ROS) and supports a role for the glutathione antioxidant system in persistent pain. *P<0.05; **P<0.01, *** P<0.001.

FIG. 4. Decreased Mitofusin 2 (MFN2) in CIPN linked to impaired mitochondrial function-reversed by 8-AG. There is evidence that impairment in MFN2 or decreased MFN2 levels may be associated with defects in degradation in cellular components (termed autophagy) resulting in accumulation of damaged organelles as reported in CIPN animal models. Decreased MFN2 levels in CIPN animal models have been associated with reduced anterograde trafficking of mitochondria necessary for proper neural transmission and synaptic function. A decrease in MFN2 expression was observed in peripheral nerves, DRG neurons as well as hindpaw epidermis (the latter shown in FIG. 4—reversed by 8-AG treatment). *P<0.05.

FIG. 5. CIPN elevates glutathione peroxidase activity (GPx) considered the ‘master antioxidant’ in rat epidermis—normalized by 8-AG. It was found that CYP—CIPN in rats elevates GPx activity in rat peripheral nerves as well as within the epidermis which is significantly decreased by 8-AG treatment. Increases in GPx activity after nerve injury is crucial to protect the nervous tissue against augmented levels of free radicals (i.e., ROS) and supports a role for the glutathione antioxidant system in persistent pain.

FIG. 6. Evidence of significant elevation in rat hindpaw epidermis of protein carbonylation. Protein carbonylation a post-translational modification induced by excessive ROS and often used as a marker of increased oxidative stress. 8-AG treatment of CYP—CIPN rats completely prevents protein carbonylation resulting in control levels (FIG. 6). *P<0.05; **P<0.01.

FIGS. 7A and 7B. CIPN alters purine metabolism that is normalized by 8-aminoguanine (8-AG) treatment. The data show that CYP—CIPN is associated with a significant decrease in levels of protective purines inosine (FIG. 7A) and guanosine (FIG. 7B) in the epidermis (footpad). Inosine is a purine nucleoside with multiple roles and has been shown to produce anti-inflammatory and tropic effects that can protect against neural injury and may even accelerate neural regeneration. Further, there is increasing evidence that guanosine has a number of effects including stimulation of nerve regeneration, protects against apoptosis and can stimulation nerve regeneration. This data shows that treatment of CIPN rats with 8-AG (PNPase inhibitor) normalizes these levels to that of a control rat (FIG. 7A/B). *P<0.05; **P<0.01.

FIG. 8. A key regulator of neural axon growth/regeneration is significantly decreased in CIPN epidermis- and normalized by 8-AG. It was found that CYP—CIPN in rats results in a significant decrease in an enzyme termed Mst3b, a purine-sensitive Ste20-like protein kinase that plays a key role in regeneration of nerve fibers (both in the central and peripheral nervous system). Remarkably, 8-AG treatment completely restored Mst3b expression in the rat epidermis (this enzyme is sensitive to the purine nucleoside inosine) (FIG. 8), **P<0.01.

FIGS. 9A and 9B. A: CYP—CIPN rats exhibit an increase in sensitivity to mechanical stimuli which is normalized to control conditions by 8-AG treatment. B: CYP—CIPN is associated with decreased PGP-9.5 expression in hindpaw epidermis; normalized by 8-AG treatment. *P<0.05; **P<0.01.

FIGS. 10A-10C. Effect of oral 8-aminoguanine (8-AG) treatment on neuronal marker PGP 9.5 distribution in rat epidermis in CYP—CIPN (cyclophosphamide-chemotherapy-induced peripheral neuropathy). Shown are representative fluorescent images (in three separate tissue samples in each rat) of the pan-neuronal marker PGP 9.5 in Control (10A; representative image; n=3 each), CYP—CIPN (10B; representative image; n=3 each) and 8-AG-treated CYP—CIPN (10C; representative image; n=3 each) rat epidermis. Note in panel 10B that CYP—CIPN resulted in a ragged/damaged appearance of fine nerve terminals (which often do not reach to the epithelial surface). Neural damage was normalized with 8-AG treatment (10C). Magnification bars 20 mm.

FIG. 11. Effect of oral 8-aminoguanine (8-AG) treatment on mitochondrial respiration in rat spinal cords subjected to CYP—CIPN (cyclophosphamide-chemotherapy-induced peripheral neuropathy). Mitochondrial respiration was measured using a Clark-type oxygen electrode on spinal cord (L3-L5) homogenates (isolated mitochondria). CYP—CIPN reduced the respiratory control ratio (RCR) and this effect was prevented by treatment with 8-AG (Control, CYP and CYP+8-AG). Neuronal survival is linked with mitochondrial homeostasis. RCRs were obtained in multiple spinal cord samples (11-27) from each animal (n=3-5 per group) and the RCRs from each animal were averaged. Asterisks (****) indicate p<0.0001.

FIG. 12. Effect of oral 8-aminoguanine (8-AG) treatment on tactile sensitivity in rat epidermis. PAC-CIPN (paclitaxel-chemotherapy-induced peripheral neuropathy) increased sensitivity to mechanical stimuli applied to the hindpaw footpad (using von Frey method), which was normalized by 8-AG treatment (Control, n=4; PAC, n=8; PAC+8AG, n=8; with multiple assays performed per animal). These results indicate that 8-AG treatment normalizes behavioral changes to a control state. Statistical significance was tested with one-way ANOVA followed by Tukey posthoc test. Asterisks (**) indicates p<0.01.

FIG. 13. Effect of oral 8-aminoguanine (8-AG) treatment on protein carbonylation in rat epidermis. PAC-CIPN (paclitaxel-chemotherapy-induced peripheral neuropathy) increased protein carbonylation in the rat epidermis. 8-AG significantly reduced protein carbonylation (n=6 control; n=4 PAC; n=4 PAC+8-AG) in PAC-CIPN rat epidermis. Protein carbonylation is a post-translational modification induced by excessive reactive oxygen species (ROS) and often used as a biomarker for increased oxidative stress. Statistical significance was tested with one-way ANOVA followed by Tukey posthoc test. Asterisks (**) indicate p<0.01.

FIG. 14. Effect of oral 8-aminoguanine (8-AG) treatment on the macrophage biomarker CD68 in rat spinal cord. PAC-CIPN (paclitaxel-chemotherapy-induced peripheral neuropathy) increased CD68 expression in the rat spinal cord. 8-AG significantly reduced CD68 expression (n=6 control; n=4 PAC; n=4 PAC+8-AG) in PAC-CIPN rat spinal cord. Increased density of CD68 effector macrophages has been associated with neuropathic pain models and depletion of these pro-inflammatory macrophages has been shown to reduce sensitivity to neuropathic pain. Statistical significance was tested with one-way ANOVA followed by Tukey posthoc test. Asterisks (*) indicate p<0.05, (***) indicate p<0.001.

FIGS. 15A-15C. Effect of oral 8-aminoguanine (8-AG) treatment on microglial activation. CYP—CIPN rats are associated with increased expression of IBA-1, a calcium-binding protein expressed in microglia (FIG. 15A). IBA-1 is upregulated during activation of these cells (FIG. 15B, measured by densitometry) (control n=15; CYP n=19; CYP+8-AG n=14). These changes are both normalized by treatment of CYP—CIPN rats with a PNPase inhibitor 8-AG (FIGS. 15A and 15B). Given the role of microglia in both somatic and visceral pain conditions, these findings suggest that inhibition of PNPase (highly expressed in microglia, FIG. 15C), can reverse functional and morphological changes detected in neuropathic pain. Statistical significance was tested with one-way ANOVA followed by Tukey posthoc test. Asterisks (*) indicates p<0.05.

FIG. 16. Effect of oral 8-aminoguanine (8-AG) treatment on tactile sensitivity in rat epidermis. Peripheral nerve injury increases sensitivity to mechanical stimuli applied to the hindpaw footpad (using von Frey method) which was measured at day 2 and day 5 post injury. These outcome measures were normalized by 8-AG treatment (Control, n=4; Injury, n=4; Injury+8AG, n=4; with multiple assays performed per animal). These results indicate that 8-AG treatment normalizes behavioral changes to that of a control state. Statistical significance was tested with one-way ANOVA followed by Tukey posthoc test. Asterisks (**) indicates p<0.01.

FIG. 17. Effect of oral 8-aminoguanine (8-AG) treatment on peripheral nerve injury-induced alteration in superoxide dismutase (SOD1) in rat epidermis. Sciatic nerve injury (SNI) decreased SOD1 expression in the rat epidermis which was normalized by 8-aminoguanine (8-AG) treatment (SNI+8-AG). SOD1 is an enzyme that breaks down potentially harmful oxygen molecules and thus is considered an important antioxidant defense in nearly all cells. The reduction of SOD1 by sciatic nerve injury in rat epidermis may trigger excessive levels of tissue damaging hydroxyl radicals contributing to nerve damage. The prevention of SNI-induced alteration of SOD1 by 8-AG suggests that 8-AG protects against neuropathy induced by nerve injury. Statistical significance was tested with one-way ANOVA followed by Tukey posthoc test. Asterisks (*) indicate p<0.05.

### DETAILED DESCRIPTION OF SEVERAL EMBODIMENTS

Disclosed herein is the use a PNPase inhibitor or a PNPase purine nucleoside substrate to treat a peripheral neuropathy and to improve peripheral nerve function in a subject with a peripheral neuropathy. These methods include selecting the subject with the peripheral neuropathy; and administering to the subject a therapeutically effective amount of a purine nucleoside phosphorylase (PNPase) inhibitor and/or a PNPase purine nucleoside substrate. Methods for treating a peripheral neuropathy are disclosed herein. Methods are also disclosed for improving nerve function in a subject with a peripheral neuropathy.

### I. Terms

Unless otherwise noted, technical terms are used according to conventional usage. Definitions of common terms in molecular biology can be found in Krebs et al (Eds.), Lewin's Genes XII, published by Jones & Bartlett Publishers, 2017; and Meyers et al. (eds.), The Encyclopedia of Cell Biology and Molecular Medicine, published by Wiley-VCH in 16 volumes, 2008; and other similar references.

As used herein, the singular forms “a,” “an,” and “the” refer to both the singular as well as plural unless the context clearly indicates otherwise. Further, compositions of use in the methods herein can be used alone or in combination. Although many methods and materials similar or equivalent to those described herein can be used, particular suitable methods and materials are described below. The term “comprises” means “includes.” The term “about” means within five percent, unless otherwise indicated. All publications, patent applications, patents, and other references mentioned herein are incorporated by reference in their entirety. In case of conflict, the present specification, including explanations of terms, will control. In addition, the materials, methods, and examples are illustrative only and not intended to be limiting. To facilitate review of the various embodiments, the following explanations of terms are provided.

Administration: To provide or give a subject an agent (such as a PNPase transition state analog or a guanine, guanosine, inosine, or hypoxanthine comprising a substituent at the 8-position) by any effective route. Exemplary routes of administration include, but are not limited to, direct administration (such as via ocular delivery), topical administration (such as on the surface of the eye), oral, injection (such as subcutaneous, intramuscular, intradermal, intraperitoneal, intravenous, and intratumoral), sublingual, rectal, transdermal, intranasal, vaginal, and inhalation routes.

Agent: Any polypeptide, compound, small molecule, organic compound, salt, polynucleotide, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease, such as, but not limited to, peripheral neuropathy.

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atoms to 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (such as cycloalkenyl), cis, or trans (such as E or Z).

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (such as alkane). An alkyl group can be branched, straight-chain, or cyclic (such as cycloalkyl).

Alkoxyl: A univalent radical R—O—, or anion R—O—, wherein R is an alkyl group.

Alkynyl: An unsaturated monovalent hydrocarbon having at least two carbon atoms to 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (such as cycloalkynyl).

Amide: —NC(O)R, wherein R is hydrogen, aliphatic, aryl, heteroaliphatic, or heteroaryl.

Amine: —NR′R, wherein each of R and R′ independently are hydrogen, aliphatic, aryl, heteroaliphatic, or heteroaryl.

Analog: A compound with a molecular structure closely similar to that of another, such as an analog of the transition state of a substrate during catalysis (for example, a transition state analog of catalysis by PNPase).

Animal: Living multi-cellular vertebrate organisms, a category that includes, for example, mammals and birds. The term mammal includes both human and non-human mammals. Similarly, the term “subject” includes both human and veterinary subjects.

Aryl: An aromatic carbocyclic group comprising at least five carbon atoms to 15 carbon atoms, such as five to ten carbon atoms, having a single ring or multiple condensed rings, which condensed rings can or may not be aromatic provided that the point of attachment is through an atom of the aromatic carbocyclic group.

Carbonate: —OC(O)OR, wherein R is hydrogen, aliphatic, aryl, heteroaliphatic, or heteroaryl.

Carbonyl: C═O, wherein the carbon located at the 8 position of guanine or guanosine forms a double bond with an oxygen atom.

Carboxyl: —C(O)OR, wherein R is hydrogen, aliphatic, aryl, heteroaliphatic, or heteroaryl.

Cluster of Differentiation (CD)68: A protein highly expressed by cells in the monocyte lineage (e.g., monocytic phagocytes, osteoclasts), by circulating macrophages, and by tissue macrophages (e.g., Kupffer cells, microglia) in mammals. Human CD68 is a transmembrane glycoprotein that has a molecular weight of 110 kD. It is 354 amino acids in length, with predicted molecular weight of 37.4 kD (without glycosylation). An exemplary nucleic acid sequence encoding human CD68 is presented in GENBANK® Accession No. NM_001251.3, Dec. 12, 2021, incorporated by reference herein, and an exemplary amino acid sequence of human CD68 is presented in GENBANK® Accession No NP_001242, Dec. 12, 2021, incorporated by reference herein.

Complex regional pain syndrome: A form of chronic pain that usually affects an arm or a leg. CRPS typically develops after an injury, a surgery, a stroke or a heart attack. The pain is out of proportion to the severity of the initial injury. Diagnosis is usually based on an exam and medical history.

Control subject: A control subject is a subject that is used to provide a basis for comparison. As a comparison to subjects with or at risk for a particular condition (such as a subject that has a peripheral neuropathy), control subjects may belong to a group of healthy subject who are studied to observe how their symptoms, traits, or behaviors compare to a group of subjects with or at risk for a particular condition.

Cytokines: A broad category of small proteins (approximately 5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumor necrosis factors but generally not hormones or growth factors. Cytokines are produced by a broad range of cells, including immune cells, such as macrophages, B lymphocytes, T lymphocytes, and mast cells, as well as endothelial cells, fibroblasts, and various stromal cells; a given cytokine may be produced by more than one type of cell. Cytokines are important in health and disease, specifically in host responses to infection, immune responses, inflammation, trauma, sepsis, cancer, and reproduction. They act through receptors and are especially important in the immune system; cytokines modulate the balance between humoral and cell-based immune responses, and they regulate the maturation, growth, and responsiveness of particular cell populations. Cytokines include interleukins, such as IL-1beta, and chemoattractants, such as monocyte chemoattractant protein-1 (MCP-1).

Ester: —OC(O)R, wherein R is hydrogen, aliphatic, aryl, heteroaliphatic, or heteroaryl.

Glutathione Peroxidase: A cytosolic enzyme that catalyzes the reduction of hydrogen peroxide to water and oxygen as well as catalyzing the reduction of peroxide radicals to alcohols and oxygen. Glutathione (GSH) is an antioxidant in plants, animals, fungi, and some bacteria and archaea; glutathione disulfide (GSSG) is a disulfide derived from two GHS molecules. The activity of glutathione peroxidase is shown in the formula below:

H₂O₂+2GSH→2H₂O+GSSS

Halogen: bromo, fluoro, iodo, or chloro.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroaryl: An aryl group comprising at least one heteroatom to six heteroatoms, such as one to four heteroatoms, which can be selected from, but not limited to oxygen, nitrogen, sulfur, selenium, phosphorous, and oxidized forms thereof within the ring. Such heteroaryl groups can have a single ring or multiple condensed rings, wherein the condensed rings may or may not be aromatic or contain a heteroatom, provided that the point of attachment is through an atom of the aromatic heteroaryl group.

Hydroxyl: —OH.

Mitochondrial Respiration: The set of metabolic reactions and processes requiring oxygen that takes place in mitochondria to convert the energy stored in macronutrients to adenosine triphosphate (ATP), the universal energy donor in the cell. A dominant role for the mitochondria is the production of ATP, performed by oxidizing the major products of glucose: pyruvate, and NADH, through a process termed oxidative phosphorylation.

Mitofusin (MFN) 2: Mitofusins are GTPases embedded in the outer membrane of the mitochondria. MFN1 and MFN2 are homologs proteins that belong to the large family of mitochondrial transmembrane GTPases. MFN1 and MFN 2 are highly similar proteins (˜80% similarity in humans), consisting of 737 and 757 amino acids, respectively. They have a cytosolic, N-terminal GTPase domain, sequentially followed by a spacer, a first coiled-coil heptad-repeat (HR1) domain, a spacer, two very close transmembrane domains (TM) crossing the OMM, a spacer and a second, C-terminal heptad-repeat domain (HR2). Exemplary nucleic acid and amino acid sequences for human MFN2 can be found in GENBANK® Accession No. NM_001127660.2, Sep. 19, 2021, incorporated herein by reference.

Neuropathic postural orthostatic tachycardia syndrome (POTS): A form of dysautonomia that relates to the position of the body, wherein there is an increased heart rate when standing upright. Symptoms usually results from a combination of low blood in the circulation, pooling of blood below the level of the heart when upright, and elevated of levels of hormones such as epinephrine and norepinephrine. In neuropathic POTS there generally is small fiber nerve damage.

Nitro: —NO₂.

Nitroso: —NO.

Peripheral Nerve: An enclosed cable-like axonal bundle in the peripheral nervous system. A nerve provides a common pathway for the electrochemical nerve impulses (action potentials) that are transmitted along each of the axons to peripheral organs or, in the case of sensory nerves, from the periphery back to the central nervous system. Each axon, within the nerve, is an extension of an individual neuron along with other supportive cells such as some Schwann cells that coat the axons in myelin. Nerves that transmit signals from the brain are called motor nerves or efferent nerves, while those nerves that transmit information from the body to the central nervous system (CNS) are called sensory nerves or afferent nerves. Spinal nerves serve both functions and are called mixed nerves. Motor neurons have cell bodies located in the motor cortex, brainstem or spinal cord and directly or indirectly control organs, such as the muscles and the glands. The peripheral nervous system (PNS) is divided into three separate subsystems, the somatic, autonomic, and enteric nervous systems. A somatic nerve mediates voluntary movement. An improvement in peripheral nerve function includes an improved response to a sensory stimulation or improved motor neuron function. This can result in improved response(s) to stimuli.

In animals, tests are used to assess sensory function that measure responses and/or reaction time to various stimuli. Examples include devices that measure animal's response to heat and those that measure animal's response to mechanical pressure (e.g., von Frey filament testing). Motor coordination can be assessed using a RotaRod apparatus. Other methods include electromyography (EMG) and motor and sensory nerve conduction velocity (MNCV and SNCV).

In patients, a general neurologic exam is typically done along with and examination portions of questionnaires such as the Utah Early Neuropathy Scale, the Neuropathy Impairment Neuropathy Score-Lower leg (NIS-LL) and the chemotherapy-induced peripheral neuropathy questionnaire (QLQ-CIPN20). This also can include laboratory tests (electromyography—EMG—with nerve conduction studies and skin biopsies to evaluate cutaneous nerve innervation).

The autonomic nervous system is further subdivided into the sympathetic and the parasympathetic nervous systems. The sympathetic nervous system is activated in cases of emergencies to mobilize energy, while the parasympathetic nervous system is activated when organisms are in a relaxed state. The enteric nervous system functions to control the gastrointestinal system. Both autonomic and enteric nervous systems function involuntarily.

Peripheral Neuropathy: Damage to the peripheral nervous system, that impairs sensation, movement, gland or organ function depending on which nerves are affected. The motor, sensory and/or autonomic nerves can be affected. Peripheral neuropathy can be acute or chronic. Common causes include systemic diseases (such as diabetes or leprosy), hyperglycemia-induced glycation, and vitamin deficiency. Peripheral neuropathy also can be cause by medication, such as from treatment with chemotherapy, or antibiotics, including metronidazole and the fluoroquinolone class of antibiotics (such as ciprofloxacin, levofloxacin, moxifloxacin)). Peripheral neuropathy also can be cause by traumatic injury, radiation therapy, excessive alcohol consumption, immune system disease, celiac disease, non-celiac gluten sensitivity, and viral infection. There are also genetic causes of peripheral neuropathy. Peripheral neuropathy can be idiopathic.

Neuropathy affecting just one nerve is termed “mononeuropathy” and neuropathy involving nerves in the same areas on both sides of the body is called “symmetrical polyneuropathy” or “polyneuropathy”. When two or more (typically just a few, but sometimes many) separate nerves in disparate areas of the body are affected, the condition can be called mononeuritis multiplex, multifocal mononeuropathy, or multiple mononeuropathy.

Pharmaceutically acceptable carrier: As used herein, the term “pharmaceutically acceptable carrier” includes any and all solvents, dispersion media, coatings, surfactants, antioxidants, preservatives (such as antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, such as Remington's Pharmaceutical Sciences, 1289-1329, 1990, incorporated herein by reference). Except insofar as any conventional carrier is incompatible with the active ingredient, its use in the therapeutic or pharmaceutical compositions is contemplated.

PNPase: Purine nucleoside phosphorylase, a glycosyltransferase, is an enzyme that catalyzes a chemical reaction between purine nucleoside (such as the PNPase purine nucleoside substrates inosine and guanosine) and phosphate. PNPase inhibitors inhibit the catalytic action of a PNPase. Non-limiting examples of PNPase inhibitors include 8-substituted guanine, guanosine, inosine, and hypoxanthine compounds (for example, 8-aminoguanine, 8-aminoguanosine, 8-aminoinosine, and 8-aminohypoxanthine); and PNPase transition state analogs, such as forodesine, or a forodesine derivative, for example, DADMe-immucillin-H, DATMe-immucillin-H, or SerMe-immucillin-H, or as described in U.S. Pat. Nos. 4,985,433; 4,985,434, 5,008,265; 5,008,270; 5,565,463, and 5,721,240 as well as US pat. pub. no. 2018/0258091A1, all of which are incorporated herein by reference in their entireties).

Sensory neurons: Also known as afferent neurons, sensory neurons are nerve cells within the peripheral nervous system responsible for converting stimuli from the environment of the neuron into internal electrical impulses and transmitting the impulse to the central nervous system.

Subject: As used herein, the term “subject” refers to a mammal and includes, without limitation, humans and veterinary subjects, including domestic animals (such as dogs or cats), farm animals (such as cows, horses, or pigs), and laboratory animals (such as mice, rats, hamsters, guinea pigs, pigs, rabbits, dogs, or non-human primates (monkeys)).

Superoxide Dismutase (SOD): An enzyme that alternately catalyzes the dismutation (or partitioning) of the superoxide (O⁻₂) radical into ordinary molecular oxygen (O₂) and hydrogen peroxide (H₂O₂), in the following reaction:

2HO₂→O₂+H₂O₂

The pathway by which SOD-catalyzed dismutation of superoxide may be written, for Cu, Zn SOD, with the following reactions:

Cu²⁺—SOD+O₂⁻→Cu⁺—SOD+O₂ (reduction of copper; oxidation of superoxide)

Cu⁺—SOD+O₂⁻+2H⁺→Cu²⁺—SOD+H₂O₂ (oxidation of copper; reduction of superoxide)

The general form, applicable to all the different metal-coordinated forms of SOD, can be written as follows:

M⁽ⁿ⁺¹⁾⁺—SOD+O₂⁻→Mⁿ⁺—SOD+O₂

Mⁿ⁺—SOD+O₂⁻+2H⁺→M⁽ⁿ⁺¹⁾⁺—SOD→0H₂O₂

wherein M=Cu (n=1); Mn (n=2); Fe (n=2); Ni (n=2).

Superoxide is produced as a by-product of oxygen metabolism and, if not regulated, causes many types of cell damage. SOD is an important antioxidant defense in nearly all living cells exposed to oxygen.

Transition state analog: A chemical compound with a chemical structure that resembles the transition state of a substrate molecule in an enzyme-catalyzed chemical reaction. A ‘PNPase transition state analog’ is a compound that resembles the transition state of the reaction (such as catalysis of inosine to hypoxanthine or catalysis of guanosine to guanine) catalyzed by PNPase. These compounds act as an inhibitor of the PNPase by blocking its active site.

Therapeutically effective amount: The term “therapeutically effective amount” refers to the amount of an active ingredient (such as, but not limited to, a PNPase transition state analog, 8-substituted guanine, 8-substituted guanosine, 8-substituted guanosine, and 8-substituted inosine) that is sufficient to effect treatment when administered to a mammal that has a peripheral neuropathy. The therapeutically effective amount will vary depending upon the subject and disease condition being treated, the weight and age of the subject, the severity of the disease condition, the manner of administration and the like, which can readily be determined by a prescribing physician.

Treating or inhibiting a disease: Inhibiting the full development of a disease or condition, for example, in a subject who has, or is at risk for, a disease such as a peripheral neuropathy. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, an improvement in the overall health or well-being of the subject, or by other parameters well known in the art that are specific to the particular disease. “Prophylaxis” is a treatment administered to a subject who does not exhibit signs of a disease or exhibits only early signs for the purpose of decreasing the risk of developing pathology.

### II. Overview

Methods for using a PNPase inhibitor or a PNPase purine nucleoside substrate are disclosed herein, such as for treating a peripheral neuropathy. These methods include selecting the subject with the peripheral neuropathy; and administering to the subject a therapeutically effective amount of a purine nucleoside phosphorylase (PNPase) inhibitor and/or a PNPase purine nucleoside substrate, thereby treating the peripheral neuropathy in the subject. Methods are also disclosed for of improving neuron function of a subject with peripheral neuropathy. These methods include selecting the subject with the peripheral neuropathy, wherein the subject is in need of improving neuron function, and administering to the subject a therapeutically effective amount of a purine nucleoside phosphorylase (PNPase) inhibitor and/or a PNPase purine nucleoside substrate. In some embodiments, the method improves sensory nerve function of the subject and/or motor neuron function in the subject.

In some embodiments, the PNPase inhibitor is a guanine comprising a substituent at the 8-position, a guanosine comprising a substituent at the 8-position, an inosine comprising a substituent at the 8-position, a hypoxanthine comprising a substituent at the 8-position, a PNPase transition state analog, or a pharmaceutically acceptable salt thereof. In some non-limiting examples, the substituent is amine, hydroxyl, nitro, nitroso, alkoxy, carbonyl, halogen, carboxyl, ester, carbonate, amide, or haloaliphatic. In some non-limiting examples, the substituent is amine. In some embodiments, the guanine comprising a substituent at the 8-position is 8-aminoguanine. In other embodiments, PNPase transition state analog is: 7-[(2S,3S,4R,5R)-3,4-dihydroxy-5-(hydroxymethyl)pyrrolidin-2-yl]-3H,4H,5H-pyrrolo[3,2-d]pyrimidin-4-one; 7-(((3R,4R)-3-hydroxy-4-(hydroxymethyl)pyrrolidin-1-yl)methyl)-3H-pyrrolo[3,2-d]pyrimidin-4(5H)-one; 7-(((2R,3S)-1,3,4-trihydroxybutan-2-ylamino)methyl)-3H-pyrrolo[3,2-d]pyrimidin-4(5H)-one; 7-((1,3-dihydroxypropan-2-ylamino)methyl)-3H-pyrrolo[3,2-d]pyrimidin-4(5H)-one; or a pharmaceutically acceptable salt thereof. IN more embodiments, the wherein the pharmaceutically acceptable salt is a chloride salt.

In some embodiments, the PNPase inhibitor and/or a PNPase purine nucleoside substrate is administered systemically to the subject. In some non-limiting examples, the PNPase inhibitor and/or a PNPase purine nucleoside substrate is administered orally, intravenously, or intramuscularly to the subject. In other non-limiting examples, administering comprises repeated delivering to the subject. In some embodiments, the PNPase inhibitor is the guanine comprising a substituent at the 8-position or the guanosine comprising a substituent at the 8-position. The subject can be a veterinary or a human subject.

In some embodiments, the peripheral neuropathy is induced by a toxic agent, such as a chemotherapeutic agent or radiation. In other embodiments, the toxic agent is a platinum compound (such as cisplatin, carboplatin, or oxaliplatin), a taxane (such as paclitaxel or docetaxel), a vinca alkaloid (such as vinblastine, vincristine, or vindesine), a proteosome inhibitor (such as bortezomib, or ixazomib), thalidomide, or an immune check-point inhibitor (such as PD-1 or PD-L1 inhibitors such as Pembrolizumab, Avelumab, Durvalumab). The peripheral neuropathy can be induced by cyclophosphamide. The peripheral neuropathy can be induced by combination chemotherapy, wherein more than one agent is used to treat a subject. In other embodiments, the peripheral neuropathy is genetically acquired. In further embodiments, the peripheral neuropathy is diabetic neuropathy or Guillian-Barre syndrome. In more embodiments, the peripheral neuropathy results from a systemic or infectious disease. In some embodiments, the peripheral neuropathy results from a post-surgical complication.

In some embodiments, the disclosed methods reduce superoxide dismutase levels in peripheral nerves in the subject. In other embodiments, the disclosed methods reduce mitofusion 2 levels in epidermis in the subject. In further embodiments, the disclosed methods reduce glutathione peroxidase activity and/or protein carbonylation in epidermis in the subject. In yet other embodiments, the disclosed methods reduce inosine and/or guanosine in epidermis in the subject. In more embodiments, the disclosed methods reduce nerve damage and/or edema in affected tissue the subject. In other embodiments, the disclosed methods increase epidermal sensitivity in affected tissue the subject. In some embodiments, the disclosed methods increase mitochondrial respiration in affected tissue the subject.

### III. Therapeutic Compounds, Pharmaceutical Compositions, and Methods of Administration

It is disclosed herein that a PNPase inhibitor or a PNPase purine nucleoside substrate (for example, PNPase purine nucleoside substrates that can act as both a substrate or a PNPase inhibitor) can be used therapeutically for the treatment of peripheral neuropathy.

Examples of (PNPase) inhibitor or a PNPase purine nucleoside substrates that can be used therapeutically for the treatment of peripheral neuropathy include guanine; guanosine; inosine; hypoxanthine; amiloride; an 8-substituted guanine (such as 8-AG, 8-hydroxyguanine, or 8-nitroguanine), guanosine (such as 8-aminoguanosine or 8-hydroxyguanosine), inosine (such as 8-aminoinosine), or hypoxanthine (such as 8-aminohypoxanthine); forodesine or a derivative thereof (for example, DADMe-Immucillin-H, DATMe-Immucillin-H, or SerMe-Immucillin-H, or such as described in U.S. Pat. Nos. 4,985,433; 4,985,434, 5,008,265; 5,008,270; 5,565,463, and 5,721,240 as well as US pat. pub. no. 2018/0258091A1, all of which are incorporated herein by reference in their entireties), or a pharmaceutically acceptable salt thereof (such as a chloride salt). The 8-substituted guanine, guanosine, inosine, and hypoxanthine compounds are referred to as Formula 1 (guanine with a substituent at the 8 position), Formula 2 (guanosine with a substituent at the 8 position), Formula 3 (hypoxanthine with a substituent at the 8 position), and Formula 4 (inosine with a substituent at the 8 position), respectively. The general chemical structures of these compounds are shown below and are further defined in the Terms section.

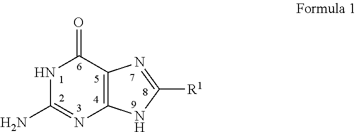

With reference to Formula 1, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 1, R¹ is amine (—NR′R), and each of R and R′ independently are hydrogen, aliphatic, aryl, heteroaliphatic, or heteroaryl.

In some embodiments of Formula 1, R¹ is carbonyl (C═O), and the carbon located at the 8 position of Formula 1 forms a double bond with an oxygen atom.

In some embodiments of Formula 1, R¹ is halogen, and the halogen is bromo, fluoro, iodo, or chloro.

In some embodiments of Formula 1, R¹ is carboxyl (—C(O)OR), and R is hydrogen, aliphatic, aryl, heteroaliphatic, or heteroaryl.

In some embodiments of Formula 1, R¹ is ester (—OC(O)R), and R is hydrogen, aliphatic, aryl, heteroaliphatic, or heteroaryl.

In some embodiments of Formula 1, R¹ is carbonate (—OC(O)OR), and R is hydrogen, aliphatic, aryl, heteroaliphatic, or heteroaryl.

In some embodiments of Formula 1, R¹ is amide (—NC(O)R), and R is hydrogen, aliphatic, aryl, heteroaliphatic, or heteroaryl.

In some embodiments of Formula 1, R¹ is haloaliphatic (—CH₂X, —CHX₂, or —CX₃), and each X independently is halogen (Cl, Br, F, or I).

Any of the compound embodiments of Formula 1 can be included in a pharmaceutical composition and used in the methods disclosed herein.

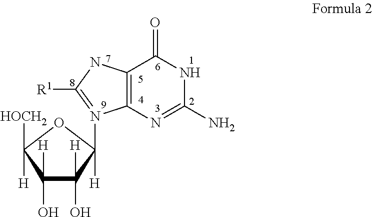

With reference to Formula 1, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 2, R¹ is amine (—NR′R), and each of R and R′ independently are hydrogen, aliphatic, aryl, heteroaliphatic, or heteroaryl.

In some embodiments of Formula 2, R¹ is carbonyl (C═O), and the carbon located at the 8 position of Formula 1 forms a double bond with an oxygen atom.

In some embodiments of Formula 2, R¹ is halogen, and the halogen is bromo, fluoro, iodo, or chloro.

In some embodiments of Formula 2, R¹ is carboxyl (—C(O)OR), and R is hydrogen, aliphatic, aryl, heteroaliphatic, or heteroaryl.

In some embodiments of Formula 2, R¹ is ester (—OC(O)R), and R is hydrogen, aliphatic, aryl, heteroaliphatic, or heteroaryl.

In some embodiments of Formula 2, R¹ is carbonate (—OC(O)OR), and R is hydrogen, aliphatic, aryl, heteroaliphatic, or heteroaryl.

In some embodiments of Formula 2, R¹ is amide (—NC(O)R), and R is hydrogen, aliphatic, aryl, heteroaliphatic, r heteroaryl.

In some embodiments of Formula 2, R¹ is haloaliphatic (—CH₂X, —CHX₂, or —CX₃), and each X independently is halogen (Cl, Br, F, or I).

Any of the compound embodiments of Formula 2 can be included in a pharmaceutical composition and used in the methods disclosed herein.

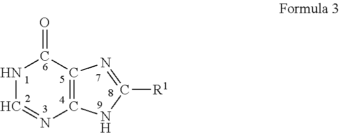

With reference to Formula 3, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 3, R^{1I} is amine (—NR′R), and each of R and R′ independently are hydrogen, aliphatic, aryl, hetero aliphatic, or heteroaryl.

In some embodiments of Formula 3, R^{1I} is carbonyl (C═O), and the carbon located at the 8 position of Formula 1 forms a double bond with an oxygen atom.

In some embodiments of Formula 3, R^{1I} is halogen, and the halogen is bromo, fluoro, iodo, or chloro.

In some embodiments of Formula 3, R¹ is carboxyl (—C(O)OR), and R is hydrogen, aliphatic, aryl, heteroaliphatic, or heteroaryl.

In some embodiments of Formula 3, R¹ is ester (—OC(O)R), and R is hydrogen, aliphatic, aryl, heteroaliphatic, or heteroaryl.

In some embodiments of Formula 3, R¹ is carbonate (—OC(O)OR), and R is hydrogen, aliphatic, aryl, heteroaliphatic, or heteroaryl.

In some embodiments of Formula 3, R¹ is amide (—NC(O)R), and R is hydrogen, aliphatic, aryl, heteroaliphatic, or heteroaryl.

In some embodiments of Formula 3, R¹ is haloaliphatic (—CH₂X, —CHX₂, or —CX₃), and each X independently is halogen (Cl, Br, F, or I).

Any of the compound embodiments of Formula 3 can be included in a pharmaceutical composition and used in the methods disclosed herein.

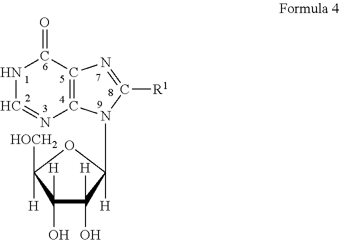

With reference to Formula 4, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 4, R¹ is amine (—NR′R), and each of R and R′ independently are hydrogen, aliphatic, aryl, heteroaliphatic, or heteroaryl.

In some embodiments of Formula 4, R¹ is carbonyl (C═O), and the carbon located at the 8 position of Formula 4 forms a double bond with an oxygen atom.

In some embodiments of Formula 4, R¹ is halogen, and the halogen is bromo, fluoro, iodo, or chloro.

In some embodiments of Formula 4, R¹ is carboxyl (—C(O)OR), and R is hydrogen, aliphatic, aryl, heteroaliphatic, or heteroaryl.

In some embodiments of Formula 4, R¹ is ester (—OC(O)R), and R is hydrogen, aliphatic, aryl, heteroaliphatic, or heteroaryl.

In some embodiments of Formula 4, R¹ is carbonate (—OC(O)OR), and R is hydrogen, aliphatic, aryl, heteroaliphatic, or heteroaryl.

In some embodiments of Formula 4, R¹ is amide (—NC(O)R), and R is hydrogen, aliphatic, aryl, heteroaliphatic, or heteroaryl.

In some embodiments of Formula 4, R¹ is haloaliphatic (—CH₂X, —CHX₂, or —CX₃), and each X independently is halogen (Cl, Br, F, or I).

Any of the compound embodiments of Formula 4 can be included in a pharmaceutical composition and used in the methods disclosed herein.

Specific compounds of use in the methods disclosed herein include those shown in Table 2.

TABLE 2
Chemical structures for compounds of use in the methods disclosed herein

| Name | Structure |
|---|---|
| Guanine | 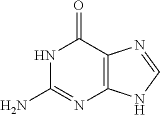 [SMILES: Nc1nc2[nH]cnc2c(=O)[nH]1] |
| Guanosine | 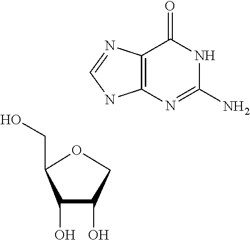 |
| 8-AG | 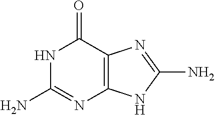 [SMILES: Nc1nc2[nH]c(N)nc2c(=O)[nH]1] |
| 8-Aminoguanosine | 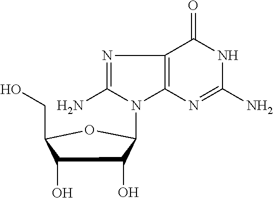 |
| 8-Hydroxyguanine | 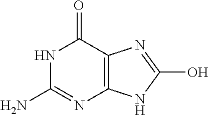 [SMILES: Nc1nc2[nH]c(O)nc2c(=O)[nH]1] |
| 8-Hydroxyguanosine | 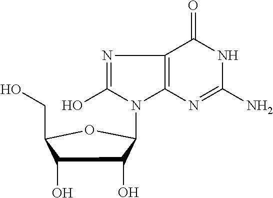 |
| 8-Nitroguanine | 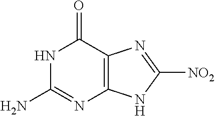 [SMILES: Nc1nc2[nH]c([N+](=O)[O-])nc2c(=O)[nH]1] |
| Amiloride | 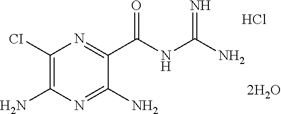 |
| Inosine | 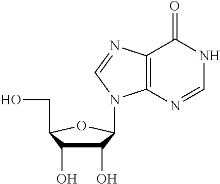 |
| Hypoxanthine | 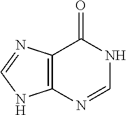 |
| 8-aminoinosine | 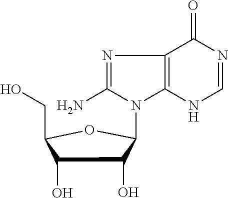 |
| 8-aminohypoxanthine | 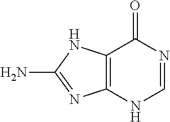 [SMILES: Nc1nc2[nH]cnc(=O)c2[nH]1] |
| Name(s) | Structure |
| Forodesine, Immucillin-H, and 7-[(2S,3S,4R,5R)-3,4- dihydroxy-5-(hydroxymethyl)pyrrolidin-2-yl]-3H,4H,5H- pyrrolo[3,2-d]pyrimidin-4-one | 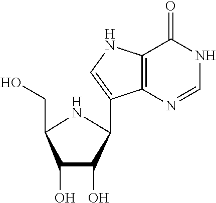 |
| DADMe-Immucillin-H, Ulodesine, and 7-(((3R,4R)-3- hydroxy-4-(hydroxymethyl)pyrrolidin-1-yl)methyl)-3H- pyrrolo[3,2-d]pyrimidin-4(5H)-one | 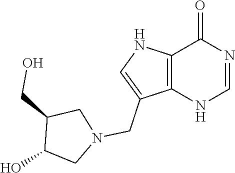 [SMILES: O=c1nc[nH]c2c(CN3C[C@H](CO)[C@@H](O)C3)c[nH]c12] |
| DATMe-Immucillin-H and 7-(((2R,3S)-1,3,4- trihydroxybutan-2-ylamino)methyl)-3H-pyrrolo[3,2- d]pyrimidin-4(5H)-one | 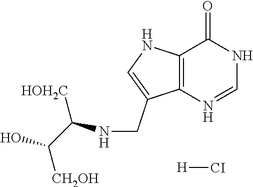 |

Compounds of use in the disclosed method include PNPase transition state analogs. As disclosed in U.S. Pat. No. 5,721,240, incorporated herein by reference in its entirety, 9-arylmethyl-substituted purines (including guanines) have been reported as PNP inhibitors in U.S. Pat. No. 4,772,606. PNPase inhibitory data cited in Drugs of the Future 13, 654 (1988) and Agents and Actions 21, 253 (1987) indicate that the 9-arylmethyl (Ar) substituted guanine derivatives of the formula:

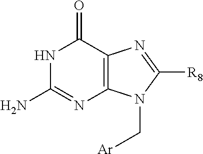

wherein R₈ represents hydrogen are markedly less potent PNP inhibitors than the corresponding compounds wherein R₈ represents amino (8-AGs).

This U.S. patent disclosed inhibitors of the formula:

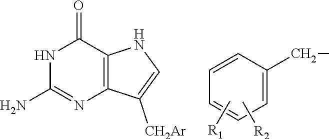

wherein CH₂Ar represents:

which R₁ represents hydrogen, halogen, C₁-C₃-alkyl, C₁-C₃-alkoxy, benzyloxy, hydroxy or trifluoromethyl; and R represents hydrogen, halogen, C₁-C₃-alkyl, C₁-C₃-alkoxy, benzyloxy, hydroxy or trifluoromethyl; provided that R₂ represents hydrogen or C₁-C₃-alkyl if R₁ represents trifluoromethyl, or that R₁ represents hydrogen or C₁-C₃-alkyl if R₂ represents trifluoromethyl; or wherein CH₂Ar represents:

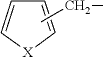

in which X represents sulfur or oxygen and in which attachment to the thiophene or furan ring is at the 2- or 3-position; and tautomers thereof.

This U.S. patent also discloses compounds specified by formulas II, III, and IV, which can also be used in the presently disclosed methods. Pharmaceutically acceptable salts and hydrates are also disclosed. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

PCT Publication No. WO99/19338, incorporated herein by reference, discloses a compound genus as a new class of inhibitors of nucleoside metabolism, including forodesine, all of which can be used in the presently disclosed methods. PCT Publication No. WO 2016/110527, also incorporated herein by reference discloses methods for synthesis of forodesine. Forodesine, also known as immucillin-H and 7-[(2S,3S,4R,5R)-3,4-dihydroxy-5-(hydroxymethyl)-2-pyrrolidinyl]-1,5-dihydropyrrolo[2,3-e]pyrimidin-4-one, is an inhibitor of purine nucleoside phosphorylase.

In specific, non-limiting examples, the transition state analog can be forodesine (also known as immucillin-H and 7-[(2S,3S,4R,5R)-3,4-dihydroxy-5-(hydroxymethyl)pyrrolidin-2-yl]-3H,4H,5H-pyrrolo[3,2-d]pyrimidin-4-one) as well as derivatives thereof, such as DADMe-immucillin-H (also known as ulodesine and 7-(((3R,4R)-3-hydroxy-4-(hydroxymethyl)pyrrolidin-1-yl)methyl)-3H-pyrrolo[3,2-d]pyrimidin-4(5H)-one), DATMe-immucillin-H (also known as 7-(((2R,3S)-1,3,4-trihydroxybutan-2-ylamino)methyl)-3H-pyrrolo[3,2-d]pyrimidin-4(5H)-one), SerMe-immucillin-H (also known as 7-((1,3-dihydroxypropan-2-ylamino)methyl)-3H-pyrrolo[3,2-d]pyrimidin-4(5H)-one). Compounds of use are disclosed in U.S. Pat. Nos. 4,985,433; 4,985,434, 5,008,265; 5,008,270; 5,565,463, and 5,721,240 as well as U.S. Published Application No. 2018/0258091A1, all of which are incorporated herein by reference in their entireties. Pharmaceutically acceptable salts of these compounds are also of use, such as, but not limited to, chloride salts. Other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

Any of these compounds can be included in pharmaceutical compositions and used in the methods disclosed herein. These pharmaceutic compositions can be formulated for systemic or local delivery for use in the presently disclosed methods.

### IV. Pharmaceutical Compositions and Use to Treat Peripheral Neuropathy

Pharmaceutical compositions that include a PNPase inhibitor, such as, but not limited to, 8-substituted guanine, 8-substituted guanosine, 8-substituted inosine, or 8-substituted hypoxanthine (for example, 8-AG, 8-aminoguanosine, 8-hydroxyguanine, 8-hydroxyguanosine, 8-nitroguanine, 8-aminoinosine, or 8-aminohypoxanthine); amiloride; a PNPase transition state analog, such as forodesine or a forodesine derivative, for example, DADMe-immucillin-H, DATMe-immucillin-H, or SerMe-immucillin-H; and/or a PNPase purine nucleoside substrate (such as guanosine and inosine, which can also act as a PNPase inhibitor); or a pharmaceutically acceptable salt thereof (such as a chloride salt, for example, a PNPase transition state analog chloride salt) can be formulated with an appropriate pharmaceutically acceptable carrier and used in the methods disclosed herein. The dosage form of the pharmaceutical composition will be determined by the mode of administration chosen. For instance, in addition to injectable fluids, topical, inhalation, oral, infusion, and suppository formulations can be employed. Oral formulations can be liquid (such as syrups, solutions, or suspensions) or solid (such as powders, pills, tablets, or capsules). Suppository preparations can also be solid, gel, or in a suspension form. Infusion preparations, administered by catheter, are generally administered as liquids. Inhalation preparations can be liquid (such as solutions or suspensions) and include mists, sprays, and the like. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art. In some embodiments, the pharmaceutical composition can be formulated for oral, intramuscular, or intravenous administration.

The amount of PNPase inhibitor or PNPase purine nucleoside substrate administered will be dependent on the subject being treated, the severity of the affliction, and the manner of administration and is best left to the judgment of the prescribing clinician. Within these bounds, the formulation to be administered will contain a quantity of the active component(s) in amounts effective to achieve the desired effect in the subject being treated. A therapeutically effective amount of PNPase inhibitor or PNPase purine nucleoside substrate can be the amount of PNPase inhibitor or PNPase purine nucleoside substrate that is necessary to treat or lower the risk of a subject for a particular disease condition (see below). In some embodiments, the administration results in improved nerve function, such as sensory and/or motor neuron function.

The pharmaceutical compositions that include PNPase inhibitor (such as 8-substituted guanine, 8-substituted guanosine, 8-substituted inosine, 8-substituted hypoxanthine, amiloride, transition state analogs, or pharmaceutically acceptable salts thereof, for example, a transition state analog chloride salt) or PNPase purine nucleoside substrate (such as guanosine and inosine, which can also act as a PNPase inhibitor) can be formulated in unit dosage form, suitable for individual administration of precise dosages. A variety of dosages and dosing regimens are possible (for example, Kilpatrick et al., International Immunopharmacology, 3:541-548, 2003; Gandhi et al., Blood, 106(13):4253-4260, 2005, both of which are incorporated herein by reference in their entireties). In one specific, non-limiting example, a unit dosage (such as intravenous dosage) can contain about 1-50 μmoles/kg, such as about 1-5, 5-10, 10-20, 20-30, 30-40, or 40-50 μmoles/kg or about 33.5 μmoles/kg of a PNPase inhibitor or a PNPase purine nucleoside substrate. In other examples, a therapeutically effective amount of a PNPase inhibitor or a PNPase purine nucleoside substrate (such as oral dosage) is about 0.1-50 mg/kg, such as about 0.1-1, 1-5, 5-10, 5-20, 10-20, 20-30, 30-40, or 40-50 mg/kg or at least about 5, 10, 20, or 30 mg/kg (such as about 5-20 mg/kg/day). In further examples, a therapeutically effective amount of a PNPase inhibitor or a PNPase purine nucleoside substrate is about 0.1-50 mg/kg, such as about 0.1-1, 1-5, 5-10, 10-20, 20-30, 30-40, or 40-50 mg/kg or at least about 0.9, 4.4, or 8.8 mg/kg. In additional examples, a therapeutically effective amount of a PNPase inhibitor or a PNPase purine nucleoside substrate is about 1-10,000 μM, such as about 1-5, 1-10, 1-100, 1-500, 100-500, 10-1,000, or 100-10,000 μM or at least about 1, 10, 25, 50, 100, 500, 750, 1,000, 5,000, or 10,000 μM, such as about 10-1,000 μM. In specific non-limiting examples, 10-1,000 μM PNPase inhibitor or a PNPase purine nucleoside substrate can be used.

Pharmaceutically acceptable salts and hydrates are also disclosed. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

A variety of administration regimens are possible (for example, Kilpatrick et al., International Immunopharmacology, 3:541-548, 2003; Gandhi et al., Blood, 106(13):4253-4260, 2005, both of which are incorporated herein by reference in their entireties). Administration with a therapeutically effective amount can be a single administration or multiple administrations. Administration can involve daily or multi-daily or less than daily (such as weekly, monthly, etc.) doses over a period of a few days to weeks or months, or even years. In a particular non-limiting example, administration involves once daily dose or twice daily dose. The particular mode/manner of administration and the dosage regimen will be selected by the attending clinician, taking into account the particulars of the case (such as the subject, the disease, the disease state/severity involved, the particular administration, and whether the treatment is prophylactic). In specific, non-limiting examples, administration can be oral, intramuscular, or by intravenous delivery.

The dosage form of the pharmaceutical composition will be determined by the mode of administration chosen, which can be systemic or localized (such as to the afflicted area). The pharmaceutically acceptable carriers and excipients useful in this disclosure are conventional (see, for example, Remington: The Science and Practice of Pharmacy, The University of the Sciences in Philadelphia, Editor, Lippincott, Williams, & Wilkins, Philadelphia, PA, 21st Edition (2005)). For instance, parenteral formulations usually comprise injectable fluids that are pharmaceutically and physiologically acceptable fluid vehicles, such as water, physiological saline, other balanced salt solutions, aqueous dextrose, glycerol, or the like. In addition to injectable fluids, inhalational, and oral formulations can be employed. For solid compositions (such as powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example, sodium acetate or sorbitan monolaurate. Excipients that can be included are, for instance, proteins, such as human serum albumin or plasma preparations. Actual methods of preparing such dosage forms are known, or will be apparent, to those skilled in the art.

The compositions of this disclosure that include PNPase inhibitor (such as 8-substituted guanine, 8-substituted guanosine, 8-substituted inosine, 8-substituted hypoxanthine, amiloride, forodesine, or a forodesine derivative, for example, 8-AG, 8-aminoguanosine, 8-hydroxyguanine, 8-hydroxyguanosine, 8-nitroguanine, 8-aminoinosine, 8-aminohypoxanthine, DADMe-immucillin-H, DATMe-immucillin-H, or SerMe-immucillin-H) or PNPase purine nucleoside substrate (such as guanosine and inosine, which can also act as a PNPase inhibitor) can be administered to humans or other animals by any method.

In some embodiments, site-specific administration of the composition can be used. For example, PNPase inhibitor, PNPase purine nucleoside substrate, and/or PNPase transition state analog or pharmaceutically acceptable salt thereof (such as chloride salt) may be administered locally, and in some examples, components administered systemically may be modified or formulated to target area Local modes of administration include, by way of example, injection. In an embodiment, significantly smaller amounts of the components (compared with systemic approaches) may exert an effect when administered locally compared to when administered systemically.

In some examples, the compositions disclosed herein can be administered systemically, such as orally or parenterally, for example, intravenously, intramuscularly, intraperitoneally (i.p.), intranasally, intradermally, intrathecally, subcutaneously, via catheter, via inhalation, or via suppository. In one non-limiting example, the composition is administered orally. For oral administration, the pharmaceutical compositions can take the form of, for example, tablets or capsules prepared by conventional means with pharmaceutically acceptable excipients, such as binding agents (for example, pregelatinized maize starch, polyvinylpyrrolidone, or hydroxypropyl methylcellulose); fillers (for example, lactose, microcrystalline cellulose, or calcium hydrogen phosphate); lubricants (for example, magnesium stearate, talc, or silica); disintegrants (for example, potato starch or sodium starch glycolate); or wetting agents (for example, sodium lauryl sulfate). The tablets can be coated by methods well known in the art. Solid dosage forms for oral administration include, but are not limited to, capsules, tablets, pills, powders, and granules. In such solid dosage forms, the active compounds are mixed with at least one pharmaceutically acceptable excipient or carrier such as, but not limited to, sodium citrate or dicalcium phosphate. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives such as suspending agents (such as sorbitol syrup, cellulose derivatives, or hydrogenated edible fats); emulsifying agents (such as lecithin or acacia); non-aqueous vehicles (such as almond oil, oily esters, ethyl alcohol, or fractionated vegetable oils); and preservatives (such as methyl or propyl-p-hydroxybenzoates or sorbic acid). The preparations can also contain buffer salts, flavoring, coloring, and sweetening agents as appropriate. For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art. Oral administration includes buccal or “sub-lingual” administration via membranes of the mouth. This can be accomplished using lozenges or a chewable gum.

Pharmaceutical compositions suitable for oral administration can be presented in discrete units each containing a predetermined amount of at least one therapeutic compound useful in the present methods; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product.

For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent, or surface active/dispersing agent(s). Molded tablets can be made by molding, in a suitable machine, the powdered compound moistened with an inert liquid diluent.

Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) in a certain part of the intestinal tract, optionally, in a delayed manner. Examples of embedding compositions which can be used include polymeric substances and waxes. The active compounds can also be in micro-encapsulated form, if appropriate, with one or more of the above mentioned excipients.

Liquid dosage forms for oral administration include, but are not limited to, pharmaceutically acceptable emulsions, solutions, suspensions, syrups, teas, and elixirs. In addition to the active compounds, the liquid dosage forms can contain inert diluents commonly used in the art such as, for example, water or other solvents, solubilizing agents, and emulsifiers, such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In some embodiments, suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

A drinkable tea can also be used in the present methods. A drinkable tea may be taken in a liquid form or in a once pulverized or granulated form together with water or hot water. When it is in a powdery or granular form, the drinkable tea may be contained in a cavity of mouth before taking hot water or water like the conventional powdery or granular drinkable tea, or it may be taken after once dissolving in hot water or water. One or more components, such as a sugar, mint, or other flavor, can be added to improve taste and easiness as a drinkable drug. Teas, syrups, and elixirs can be formulated with sweetening agents, for example glycerol, sorbitol, or sucrose. Such compositions can also contain a demulcent, a preservative, and flavoring and coloring agents.

Administration can be intravenous. Optionally, the pharmaceutical composition includes a parenteral carrier, and, in some embodiments, it is a solution that is isotonic with the blood of the recipient. Examples of such carrier vehicles include water, saline, Ringer's solution, and dextrose solution. Non-aqueous vehicles, such as fixed oils and ethyl oleate, are also useful herein, as well as liposomes.

The pharmaceutical compositions may be in the form of particles comprising a biodegradable polymer or a polysaccharide jellifying or bioadhesive polymer, an amphiphilic polymer, an agent modifying the interface properties of the particles and a pharmacologically active substance. These compositions exhibit certain biocompatibility features which allow a controlled release of the active substance. (See U.S. Pat. No. 5,700,486, incorporated herein by reference in its entirety).

In some embodiments, a PNPase inhibitor or a PNPase purine nucleoside substrate is included in a controlled release formulation, for example, a microencapsulated formulation. Various types of biodegradable and biocompatible polymers can be used, and methods of encapsulating a variety of synthetic compounds, proteins, and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2nd ed., CRC Press, 2006, all of which are incorporated by reference herein in their entireties).

In other embodiments, PNPase inhibitor or PNPase purine nucleoside substrate is included in a nanodispersion system. Nanodispersion systems and methods for producing such nanodispersions are well-known to one of skill in the art. (See, for example, U.S. Pat. No. 6,780,324; U.S. Pat. Publication No. 2009/0175953, both of which are incorporated herein by reference in their entireties). For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and PNPase inhibitor or PNPase purine nucleoside substrate (such as 80/20 w/w). In some examples, the nanodispersion is prepared using the solvent evaporation method (see, for example, Kanaze et al., Drug Dev. Indus. Pharm. 36:292-301, 2010; Kanaze et al., J. Appl. Polymer Sci. 102:460-471, 2006, both of which are incorporated herein by reference in their entireties).

Many types of release delivery systems are available and known to those of ordinary skill in the art. They include polymer based systems, such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109 (incorporated herein by reference in its entirety). Delivery systems also include non-polymer systems, such as lipids, including sterols such as cholesterol, cholesterol esters and fatty acids or neutral fats, such as mono-, di-, and tri-glycerides; hydrogel release systems; silastic systems; peptide-based systems; wax coatings; compressed tablets using conventional binders and excipients; partially fused implants; and the like. Specific examples include, but are not limited to, (a) erosional systems in which a PNPase inhibitor or a PNPase purine nucleoside substrate is contained in a form within a matrix, such as those described in U.S. Pat. Nos. 4,452,775; 4,667,014; 4,748,034; 5,239,660; and 6,218,371 (all of which are incorporated by reference herein in their entireties) and (b) diffusional systems in which an active component permeates at a controlled rate from a polymer, such as described in U.S. Pat. Nos. 3,832,253 and 3,854,480 (both of which are incorporated by reference in their entireties). In addition, pump-based hardware delivery systems can be used, some of which are adapted for implantation.

Methods are disclosed herein for treating a peripheral neuropathy. The peripheral neuropathy affects the peripheral nerves of a subject, such as the motor, sensory, sensorimotor, or autonomic nerves, or a combination thereof. Any subject with a peripheral neuropathy can be selected for treatment. The peripheral neuropathy can be acute or chronic. The disclosed methods can improve the function of the somatic, autonomic, and/or enteric nerves.

Methods are disclosed for improving neuron function of a subject with peripheral neuropathy. These methods include selecting the subject with the peripheral neuropathy, and administering to the subject an amount of a purine nucleoside phosphorylase (PNPase) inhibitor, and/or a PNPase purine nucleoside substrate, effective to improve function of peripheral neurons affected with the peripheral neuropathy in the subject. The disclosed methods can be used to improve the function of motor, sensory, and/or autonomic nerves in the subject. In some embodiments, an electromyogram (EMG) and nerve conduction velocity (NCV) test can be used to assess nerve function.

In some embodiments, the disclosed methods include the use of tests of sensory function that measure responses and/or reaction time to various stimuli. Examples include devices that measure a subject's response to heat and those that measure the subject's response to mechanical pressure (e.g., von Frey filament testing). Motor coordination can be assessed using a RotaRod apparatus. In further embodiments, the method can include the use electromyography (EMG) and motor and sensory nerve conduction velocity (MNCV and SNCV).

In further embodiments, a general neurologic exam can be performed, optionally with and examination portions of questionnaires such as the Utah Early Neuropathy Scale, the Neuropathy Impairment Neuropathy Score-Lower leg (NIS-LL) and the chemotherapy-induced peripheral neuropathy questionnaire (QLQ-CIPN20). These methods also can include performing laboratory tests (such as EMG—with nerve conduction studies, skin biopsies to evaluate cutaneous nerve innervation, and other tests).

Some toxic agents that cause neurotoxicity are therapeutic drugs, antineoplastic agents, contaminants in foods or medicinal agents, and environmental and industrial pollutants. The peripheral neuropathy can be the result of exposure to a toxic agent, such as, but not limited to, a chemotherapeutic agent or radiation. Toxic agents causing peripheral neuropathy include therapeutic agents, particularly chemotherapeutic agents used for the treatment of neoplastic disease. In certain cases, peripheral neuropathy is a major complication of cancer treatment and is the main factor limiting the dosage of chemotherapeutic agents that can be administered to a patient (Macdonald, Neurologic Clinics 9:955-967 (1991)). Agents that can cause peripheral neuropathy include cisplatin, paclitaxel and vincristine (Broun, et al., Am. J. Clin. Oncol. 16:18-21 (1993); Macdonald, Neurologic Clinics 9:955-967 (1991); Casey, et al., Brain 96:69-86 (1973). Agents include, but are not limited to, a platinum compound (such as cisplatin, carboplatin, oxaliplatin), a taxane ((paclitaxel, docetaxel, cabaliztaxel), a vina alkaloid, an anti-microtubule agent, a proteasome inhibitor (bortezomib, carfilzomib, ixazomib), thalidomide, or an immune check-point inhibitor (see Staff et al., Chemotherapy-induced peripheral neuropathy: a current review. Ann Neurol 2017: 81:772-781, incorporated herein by reference). Treatment with cyclophosphamide can cause peripheral neuropathy. Subject with a peripheral neuropathy resulting from treatment with these agents can be selected for treatment with the presently disclosed methods.

In some non-limiting examples, the peripheral neuropathy can be caused by a chemotherapeutic agent such as taxol, vincristine, cisplatin, an agent used for the treatment of an infectious diseases such as streptomycin, didanosine or zalcitabine, or any other chemically toxic agent. Subject with a peripheral neuropathy resulting from treatment with these agents can be selected for treatment with the presently disclosed methods.

In some embodiments, the peripheral neuropathy is genetically acquired, results from a systemic disease, or manifests as a post-surgical complication. In specific non-limiting examples, the peripheral neuropathy can be genetically acquired, such as resulting from Charcot-Marie-Tooth syndrome. Genetically acquired neuropathies suitable for treatment by the methods and compositions described herein include, without limitation: peroneal muscular atrophy (Charcot-Marie-Tooth Disease) hereditary amyloid neuropathies, hereditary sensory neuropathy (type I and type II), porphyric neuropathy, hereditary liability to pressure palsy, Fabry's Disease, adrenomyeloneuropathy, Riley-Day Syndrome, Dejerine-Sottas neuropathy (hereditary motor-sensory neuropathy-III), Refsum's disease, ataxia-telangiectasia, hereditary tyrosinemia, anaphalipoproteinemia, abetalipoproteinemia, giant axonal neuropathy, metachromatic leukodystrophy, globoid cell leukodystrophy, and Friedrich's ataxia. Any of these subjects can be selected for treatment with the presently disclosed methods.

In a specific non-limiting example, the peripheral neuropathy results from postural orthostatic tachycardia syndrome (POTS). In another specific non-limiting example the peripheral neuropathy results from complex regional pain syndrome (CRPS). A subject can be selected for treatment that has neuropathic POTS or CRPS.

Neuropathy is a major complication of diabetes mellitus. Thus, in some embodiments, a subject with peripheral neuropathy resulting from diabetes type 1, or a subject with diabetes type 2, is selected for treatment.

In some embodiments, the subject has diabetic neuropathy pain in a limb of the upper body, such as a hand or arm, or in a limb of the lower body, such as a foot or leg. Typical symptoms of diabetic neuropathy include numbness or reduced ability to feel pain or temperature changes, tingling or burning sensation, sharp pains or cramps, increased sensitivity to touch, muscle weakness, loss of reflexes, loss of balance and coordination, and serious foot problems such as ulcers, infections, and bone and joint pain. Often, patients experience diabetic neuropathy pain in the feet and legs first, followed by hands and arms. In some embodiments, the method further comprises selecting the subject with diabetic neuropathy pain for treatment.

In neuropathy of the feet, the loss of sensory information from the foot is related to abnormal and prolonged pressure on the areas of the foot (sensory neuropathy). Motor neuropathy leads to deformity, further increasing pressure loading on the foot. In autonomic neuropathy, loss of innervation of the sweat glands results in dry skin that cracks creating an environment amenable to infection. Autonomic dysfunction contributes further by altering the distribution of micro-circulatory blood flow, directing the blood flow through shunts and away from the nutritive skin capillaries. These factors as a whole, in conjunction with foot trauma, result in skin breakdown and ulcers. These subjects can be selected for treatment.

A subject can also be selected that has another disease or disorder that results in peripheral neuropathy. Such diseases include Raynaud's Phenomenon, including CREST syndrome, autoimmune diseases such as erythromatosis, and rheumatoid diseases. Other peripheral neuropathies include the following: HIV associated neuropathy; B12-deficiency associated neuropathy; cranial nerve palsies; drug-induced neuropathy; industrial neuropathy; lymphomatous neuropathy; myelomatous neuropathy; multi-focal motor neuropathy; chronic idiopathic sensory neuropathy; carcinomatous neuropathy; acute pain autonomic neuropathy; alcoholic neuropathy; compressive neuropathy; vasculitic/ischaemic neuropathy; mono- and poly-neuropathies. In some embodiments, these subjects can be selected for treatment.

In other preferred embodiments, the neuropathy is a non-diabetic neuropathy. In other embodiments the peripheral neuropathy can result from a systemic or infectious disease such as HIV, or an infectious disease condition such as AIDS. In further embodiments, the peripheral neuropathy manifests as a post-surgical complication. In some embodiments, these subjects can be selected for treatment.

Treatment of infectious disease conditions such as post-polio syndrome or AIDS-associated neuropathy are specifically contemplated. Other peripheral neuropathies include the following: HIV associated neuropathy; B12-deficiency associated neuropathy; cranial nerve palsies; drug-induced neuropathy; industrial neuropathy; lymphomatous neuropathy; myelomatous neuropathy; multi-focal motor neuropathy; chronic idiopathic sensory neuropathy; carcinomatous neuropathy; acute pan autonomic neuropathy; alcoholic neuropathy; compressive neuropathy; vasculitic/ischaemic neuropathy; mono- and poly-neuropathies. In further embodiments, the neuropathy is due to low back pain, Guillain-Barre Syndrome, or sciatica.

The disclosed methods can be used to treat mononeuropathy. The disclosed methods can be used to treat polyneuropathy. The disclosed methods can be used to treat mononeuritis multiplex. Subjects with any of these conditions can be selected for treatment. Any of these subjects can be selected for treatment. The disclosed methods can be used to improve the function of motor, sensory, and/or autonomic nerves in the subject.

The disclosed methods can be used to treat idiopathic peripheral neuropathy. In some embodiments, a subject with idiopathic peripheral neuropathy is selected for treatment.

A variety of PNPase inhibitors or PNPase purine nucleoside substrates, such as those described herein, can be used in the methods. In some examples, a PNPase inhibitor is administered. In specific examples, a PNPase inhibitor includes guanine; guanosine; inosine; hypoxanthine; or guanine, guanosine, inosine, or hypoxanthine with a substituent at the 8-position, such as 8-substituted guanine, 8-substituted guanosine, 8-substituted inosine, or 8-substituted guanosine; a PNPase transition state analog (such as forodesine or a forodesine derivative, for example, DADMe-immucillin-H, DATMe-immucillin-H, or SerMe-immucillin-H); or a pharmaceutically acceptable salt thereof (such as a chloride salt, for example, a PNPase transition state analog chloride salt). Exemplary substituents (such as for 8-substituted guanine, 8-substituted guanosine, 8-substituted inosine, or 8-substituted guanosine) include amine, hydroxyl, nitro, nitroso, alkoxy, carbonyl, halogen, carboxyl, ester, carbonate, amide, or haloaliphatic. In specific examples, the substituent is amine. In specific, non-limiting examples, 8-substituted guanine, such as 8-AG can be used. In specific non-limiting examples, a PNPase transition state analog or a pharmaceutically acceptable salt thereof (such as a chloride salt) can be used. However, these are exemplary only. Any of the agents and pharmaceutical compositions disclosed above are of use in these methods.

A variety of administration modes can be used. In some embodiments, administration is systemic. The administration can be oral administration, intravenous administration, or intramuscular administration. However, other routes are also of use, such as administration intraperitoneally (i.p.), intranasally, intradermally, intrathecally, subcutaneously, via catheter, via inhalation, or via suppository.

In other examples, the PNPase inhibitor or a PNPase purine nucleoside substrate is administered to the subject once or more than once (such as repeatedly). In some examples, the PNPase inhibitor or PNPase purine nucleoside substrate is administered repeatedly, or one or more times (such as at least once, at least twice, at least three times, at least four times, at least five times, at least ten times, at least fifteen times, at least twenty times, at least thirty times, or more), such as one or more times daily, weekly, bimonthly, monthly, quarterly or per year. In other embodiments, the PNPase inhibitor or a PNPase purine nucleoside substrate is administered twice a day, daily, every other day, or 1, 2, 3, 4, 5, 6, or 7 times per week.

The subject can be a veterinary subject. Thus subject can be a human subject The subject (such as a human subject) can be any age, such as a child or an adult, for example, a younger adult, middle-aged adult, or older adult. For example, the subject can be at least about 1, at least about 2, at least about 5, at least about 10, at least about 12, at least about 16, at least about 18, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, or at least about 95, about 1-5, 5-10, 10-20, 20-30, 30-40, 40-50, 50-60, 60-70,70-80, 80-95, 1-20, 20-40, 40-60, 60-70, 70-80, 80-95, or at least about 50.

Generally, the disclosed methods are administered to improve nerve function in a subject with existing peripheral neuropathy. However, treatment prior to the development of the condition, such as treatment in a subject with diabetes, but without symptoms of diabetic neuropathy, or treatment of a subject administered a chemotherapeutic agent but that does not have peripheral neuropathy, or treatment of a subject with a genetic condition that does not yet have symptoms of peripheral neuropathy, is also contemplated. This is prophylactic treatment of a subject that is “at risk” of developing the peripheral neuropathy.

In some embodiments, the disclosed methods reduce superoxide dismutase levels in peripheral nerves in the subject. In other embodiments, the disclosed methods reduce mitofusion 2 levels in epidermis in the subject. In further embodiments, the disclosed methods reduce glutathione peroxidase activity and/or protein carbonylation in epidermis in the subject. In yet other embodiments, the disclosed methods reduce inosine and/or guanosine in epidermis in the subject. In more embodiments, the disclosed methods reduce nerve damage and/or edema in affected tissue the subject. In other embodiments, the disclosed methods increase epidermal sensitivity in affected tissue the subject. In some embodiments, the disclosed methods increase mitochondrial respiration in affected tissue the subject. In further embodiments, the disclosed methods normalize biomarkers for cellular injury, such as, but not limited to, increasing epidermal protein carbonylation and/or increasing expression of CD68, such as on macrophages in the spinal cord.

In some embodiments, the disclosed methods reduce superoxide dismutase levels in peripheral nerves in the subject, as compared to a control. In further embodiments, the control can be the level of superoxide dismutase in the peripheral nerves prior to treatment, or the level of superoxide dismutase in peripheral nerves of an untreated (control) subject with the same condition. In yet other embodiments, the control is a standard value. In some embodiments, the methods include measuring superoxide dismutase in a sample from the subject.

In some embodiments, the disclosed methods reduce mitofusion 2 levels in epidermis in the subject, as compared to a control. In further embodiments, the control can be the level of mitofusion 2 in the epidermis prior to treatment, or the level of mitofusion 2 in epidermis from an untreated (control) subject with the same condition. In yet other embodiments, the control is a standard value. In some embodiments, the methods include measuring mitofusion 2 in a sample from the subject.

In some embodiments, the disclosed methods reduce glutathione peroxidase activity and/or protein carbonylation in epidermis in the subject, as compared to a control. In further embodiments, the control can be the level of glutathione peroxidase activity and/or protein carbonylation, respectively, in the epidermis prior to treatment, or the level of glutathione peroxidase activity and/or protein carbonylation, respectively, in epidermis from an untreated (control) subject with the same condition. In yet other embodiments, the control is a standard value. In some embodiments, the methods include measuring glutathione peroxidase activity and/or protein carbonylation in a sample from the subject.

In yet other embodiments, the disclosed methods reduce inosine and/or guanosine in epidermis in the subject, as compared to a control. In further embodiments, the control can be the level of inosine and/or guanosine, respectively, in the epidermis prior to treatment, or the level of inosine and/or guanosine, respectively, in epidermis from an untreated (control) subject with the same condition. In yet other embodiments, the control is a standard value. In some embodiments, the methods include measuring inosine and/or guanosine in a sample from the subject.

In more embodiments, the disclosed methods reduce nerve damage and/or edema in affected tissue the subject, as compared to a control. In further embodiments, the control can be the nerve damage and/or edema in the affected tissue prior to treatment, or the nerve damage and/or edema in affected tissue from an untreated (control) subject with the same condition. In yet other embodiments, the control is a standard value. In some embodiments, the methods include measuring nerve damage and/or edema in the subject.

In other embodiments, the disclosed methods alter epidermal sensitivity in affected tissue the subject, as compared to a control. The disclosed method can decrease sensitivity to mechanical stimuli. In further embodiments, the control can be the epidermal sensitivity in the affected tissue prior to treatment, or the epidermal sensitivity in affected tissue from an untreated (control) subject with the same condition. In yet other embodiments, the control is a standard value. In some embodiments, the methods include measuring epidermal sensitivity from the subject. In some embodiments, the method decreases sensitivity to mechanical stimuli.

In some embodiments, the disclosed methods increase mitochondrial respiration in affected tissue the subject, as compared to a control. In further embodiments, the control can be mitochondrial respiration in the affected tissue prior to treatment, or the mitochondrial respiration in affected tissue from an untreated (control) subject with the same condition. In yet other embodiments, the control is a standard value. In some embodiments, the methods include measuring mitochondrial respiration in a sample from the subject.

In some embodiments, the disclosed methods increase CD68 expression in macrophages he spinal cord the subject, as compared to a control. In further embodiments, the control can be the expression of CD68 on macrophages in the spinal cord prior to treatment, or the expression of CD68 on macrophages in the spinal cord from an untreated (control) subject with the same condition. In yet other embodiments, the control is a standard value. In some embodiments, the methods include measuring the expression of CD68 in macrophages in a sample from the subject.

The disclosure is illustrated by the following non-limiting Examples.

### EXAMPLES

### Example 1

### Materials and Methods

Materials. 8-Aminoguanine (8-AG) was purchased from Toronto Research Chemicals (Toronto, Ontario, Canada).

Animals. This study employed female Harlan Sprague Dawley (HSD) rats (3-4 mo; Envigo, Indianapolis, IN) and were divided into the following groups: 1) Control, 2) Cyclophosphamide (CYP, 75 mg/kg i.p. in saline vehicle; administered on days 0, 3 and 6); 3) 8-aminoguanine (8-AG; 5 mg/kg/day in drinking water; beginning 2 weeks prior to CYP and continuing until sacrifice (day 8)). The dose of 8-AG is based on our preliminary and published experiments. Further, the water consumption/day was tracked and no variability was found (demonstrating consistency) in terms of dosing. The investigation conforms to the Guide for the Care and Use of Laboratory Animals published by the US National Institutes of Health (NIH Publication No. 85-23, revised 1996). In addition, all tissues (dorsal root ganglion, spinal cord, peripheral nerves and hindpaw epidermis) were removed from isoflurane anesthetized rats and the tissue was used for various experimental approaches as described below. At the end of tissue removal, animals were sacrificed by thoracotomy.

### Protocol 1: Determining the Pharmacokinetics of 8-Aminoguanine (8-AG).

Rats were anesthetized with urethane (Sigma-Aldrich, St. Louis, MO) at 1.2 g/kg. A continuous infusion of 0.9% sterile saline at 25 ul/min was established via a catheter inserted into the jugular vein. A second catheter to the carotid artery was established in order to collect blood samples (200 μl) which were drawn into a 1 ml syringe prefilled with 50 U heparin. Blood samples were centrifuged at 1300 g for 10 minutes and supernatant (plasma) was then quickly frozen on dry ice and stored at −80° C. until assay. Urine samples were collected by a urethral catheter, centrifuged at 416 g for 10 minutes to remove sediment, and stored as above. Baseline blood samples and urine samples were collected followed by a single bolus of 8-aminoguanine (8-AG, 5 mg/kg) administered through the jugular catheter. Blood samples were collected after 15, 30, 60, 120 and 240 minutes. Urine samples were collected at 1, 2, 4 and 6 hours. At the end of the experiment's animals were sacrificed by thoracotomy.

Purines in urine were measured by mass spectrometry using multiple reaction monitoring. Blood samples were diluted 1 to 30 with water, and heavy isotope internal standards were added to each sample. Purines were separated by reversed-phase ultra-performance liquid chromatography (Waters UPLC BEH C18 column, 1.7 μm beads; 2.1×150 mm; Milford, MA) and quantified by multiple reaction monitoring using a triple quadrupole mass spectrometer (TSQ Quantum-Ultra; ThermoFisher Scientific, San Jose, CA) with a heated electrospray ionization source. The mobile phase was a linear gradient flow rate (300 uL/min) of 1% acetic acid in water (pH, 3; mobile phase A) and 100% methanol (mobile phase B), and was delivered with a Waters Acquity ultra-performance liquid chromatographic system. The gradient (A/B) settings were: from 0 to 2 minutes, 99.6%/0.4%; from 2 to 3 minutes, to 98.0%/2.0%; from 3 to 4 minutes, to 85.0%/15.0%; from 4 to 6.5 minutes, to 99.6%/0.4%. The instrument parameters were: sample tray temperature, 10° C.; column temperature, 50° C.; ion spray voltage, 4.0 kilovolts; ion transfer tube temperature, 350° C.; source vaporization temperature, 320° C.; Q2 CID gas, argon at 1.5 mTorr; sheath gas, nitrogen at 60 psi; auxillary gas, nitrogen at 35 psi; Q1/Q3 width, 0.7/0.7 units full-width half-maximum; scan width, 0.6 units; scan time, 0.01 seconds. The following transitions (multiple reaction monitoring) were obtained: guanosine (284→152; RT=2.94 min); ¹³C₁₀, ¹⁵N₅-guanosine (299→162, RT=2.94 min); guanine (152→135; RT=1.48 min); ¹³C₂, ¹⁵N-guanine (155→138, RT=1.48 min); hypoxanthine (137→119 m/z, RT=1.86 min); ¹³C₅-hypoxanthine (142→124 m/z, RT=1.86 min); aminoguanine (167→150 m/z, RT=1.50 min); ¹³C₂¹⁵N-aminoguanine (170→153 m/z, RT=1.50 min).

### Protocol 2. Behavioral Testing, Biomarkers and Mitochondrial Respiration Von Frey Sensory Testing

Control, CYP and 8-AG-treated CYP rats were placed in an elevated chamber on a wire mesh floor and after acclimatization, calibrated von Frey microfilaments were applied to determine paw withdrawal threshold (PWT, in grams). Briefly, microfilaments (Stoelting Corp, USA) are applied with different forces perpendicularly to the plantar surface of both ipsilateral and contralateral hindpaw by increasing and decreasing stimulus intensity. The mechanical withdrawal threshold was the minimum pressure exerted (in grams) that triggered the paw withdrawal response (using the ‘up-down’ method). All animals were tested multiple times during the experimental paradigm and averages of the individual paw withdrawal response were calculated to find the rats 50% withdrawal threshold with the development of mechano-allodynia will be taken as a significant reduction in PWT (g) that failed to elicit withdrawal responses before chemotherapy treatment.

Western immunoblotting: Following the testing paradigms, rats were deeply anesthetized and tissues (e.g., epidermis, peripheral sciatic nerves, spinal cord segments (L3-L5) and dorsal root ganglia (L3-L5) were removed, and a portion of each preparation was homogenized using Lysing Matrix D in a FASTPREP® 24 instrument (MP Biomedicals, Solon, OH) in HBSS [5 mM KCl, 0.3 mM KH₂PO₄, 138 mM NaCl, 4 mM NaHO₃, 0.3 mM Na₂HCO₃, 0.3 mM Na₂HPO₄, 5.6 mM glucose and 10 mM Hepes, pH 7.4 containing complete protease inhibitor cocktail (1 tablet/10 ml, Roche, Indianapolis, IN)] and phosphatase inhibitor cocktail (Sigma, 1:100). After centrifugation (13000 rpm; 15 min at 4° C.), the membrane protein fraction was prepared by suspending the membrane pellets in lysis buffer containing 0.3 M NaCl, 50 mM Tris-HCl (pH 7.6) and 0.5% Triton X-100 and the same concentration of protease inhibitors as above. The suspensions were incubated on ice and centrifuged (13000 rpm: 15 min at 4° C.). The protein concentrations of the supernatants were determined using the Pierce BCA protein assay (Thermo Scientific, Rockford, IL). After denaturation (100° C. for 5 min) in the presence of Laemmli sample buffer, lysate from each sample was separated on a 4-15% TGX® Stain-Free SDS-PAGE gel (Bio-Rad Laboratories, Hercules, CA). As a loading control, total protein per sample was determined using Bio-Rad Stain Free SDS-PAGE gel technology. UV-activated protein fluorescence was imaged on a CHEMIDOC® MP (Bio-Rad). After proteins were transferred to polyvinylidene fluoride membranes, non-specific binding was blocked with 5% (w/v) non-fat dried milk dissolved in TBS-T (20 mM Trizma, 137 mM NaCl, 0.1% Tween-20, pH 7.6) for one hour, rinsed with TBS-T, and incubated overnight at 4° C. with primary antibody diluted in TBS-T containing 5% (w/v) milk or 5% BSA. After washing in TBS-T, the membranes were incubated with secondary antibody [sheep anti-mouse HRP (1:5000; GE Amersham, Pittsburgh, PA) or donkey anti-rabbit HRP (1:10,000; ADVANSTA®, San Jose, CA] for 1 hour in 5% (w/v) Milk TBS-T, washed, and incubated in WESTERNBRIGHT® Quantum (ADVANSTA®, San Jose, CA) and then imaged on a CHEMIDOC® MP (BioRad). The volume (intensity) of each protein species was determined and normalized to total protein using Image Lab software (Bio-Rad). Primary antibodies included SOD1 (Cell Signaling 37385S, 1:15,000, 5% BSA TBS-T), MFN2 (Abcam aB56889, 1:500, 5% Milk TBS-T), MST3b (Cell Signaling 4062S, 1:2000, 5% BSA TBS-T), and PGP9.5 (Invitrogen PA5-29012, 1:2000, 5% Milk TBS-T). Protein carbonylation was performed with an antibody that detects dinitrophenylhydrazine (DNPH)-derivatized carbonyl groups, following kit instructions (OxyBlot Protein Oxidation Detection Kit, S7150, Millipore, Burlington, MA). Glutathione peroxidase activity was assessed using a kit from Caymen Chemical (703102, Ann Arbor, MI) following manufacturer instructions. Tissue samples were homogenized in 50 mM Tris buffer, pH 7.5, containing 5 mM EDTA and 1 mM DTT. After centrifugation at 13,000 rpm for 15 minutes, supernatants were assayed for GPx activity and protein concentrations were measured by BCA assay (used to normalize GPx).

Immunocytochemistry: A portion of tissues (a 3 mm biopsy from the rat hindpaw food pad or epidermis) is placed overnight in fixative (4% PFA) and cryoprotected (30% sucrose-phosphate buffered saline). Fluorescence immunocytochemistry was used to process cross-sections (20 μm) using antibodies (validated in rat tissue) against PGP 9.5 (Invitrogen PA5-29012, neuron specific protein; 1:500 dilution). After overnight incubation with the primary antibody, sections were incubated with secondary antibody (donkey anti-rabbit ALEXA FLUOR®555, Invitrogen A-31572, 1:1000) for one hour, washed and nuclei were counterstained with DAPI (4′, 6-diamidino-2-phenylindole, Invitrogen D21490, 1:2500, 10 minutes), washed and cover slipped with PROLONG® Gold (Invitrogen P36934). Representative images were taken and within each section 3-5 fields (each of 550% m×700 μm) were imaged per region of interest.

Mitochondrial Respiration. Homogenates (peripheral nerve and/or spinal cord) were used to measure mitochondrial respiration. Mitochondria were isolated as described earlier with modifications. Briefly, after deep anesthesia with isoflurane (5%), either peripheral nerves or spinal cord segments L3-L5 (≈80 mg) were isolated by extrusion and placed in mitochondrial solution (5 mM HEPES, 125 mM KCl, 2 mM Pi, 20 μM EDTA, 5 mM MgCl₂ and 0.2 mg/ml BSA, pH 7.4). The tissue is then minced and homogenized then centrifuged (1,000 g, 10 min). Following a second centrifugation, the pellet (containing the mitochondria) was resuspended in 100 μl of mitochondrial solution, and 25-50 μl of the suspension placed in a gas-tight vessel containing a Clark-type oxygen microelectrode (MI-730/OM-4; Microelectrodes, Londonderry, NH) to measure the state 3 (succinate+ADP) and state 4 (succinate alone) respiratory rates. The electrode was calibrated considering a total amount of dissolved O₂ in aqueous solution after equilibration with air at 36° C. to be 215 μM, zeroed with sodium dithionite. The respiratory control ratio (RCR), a measure of the “tightness of coupling” between electron transport and oxidative phosphorylation, was determined from ratio of state 3 to state 4 rates of respiration. An RCR of 2-4 is considered acceptable for complex ii substrates.

Statistics: Data were analyzed in GraphPad Prism 6 (GraphPad, La Jolla, CA) using an ordinary one-way ANOVA followed by Tukey's multiple comparison tests. SOD1 was tested for statistical significance with a Kruskal-Wallis ANOVA followed by Dunn's multiple comparison tests in the sciatic nerve. All other statistical tests were ordinary one-way ANOVA followed by Tukey's multiple comparison tests. p<0.05 was considered significant. Results are expressed as means±SEM.

### Example 2

### Results

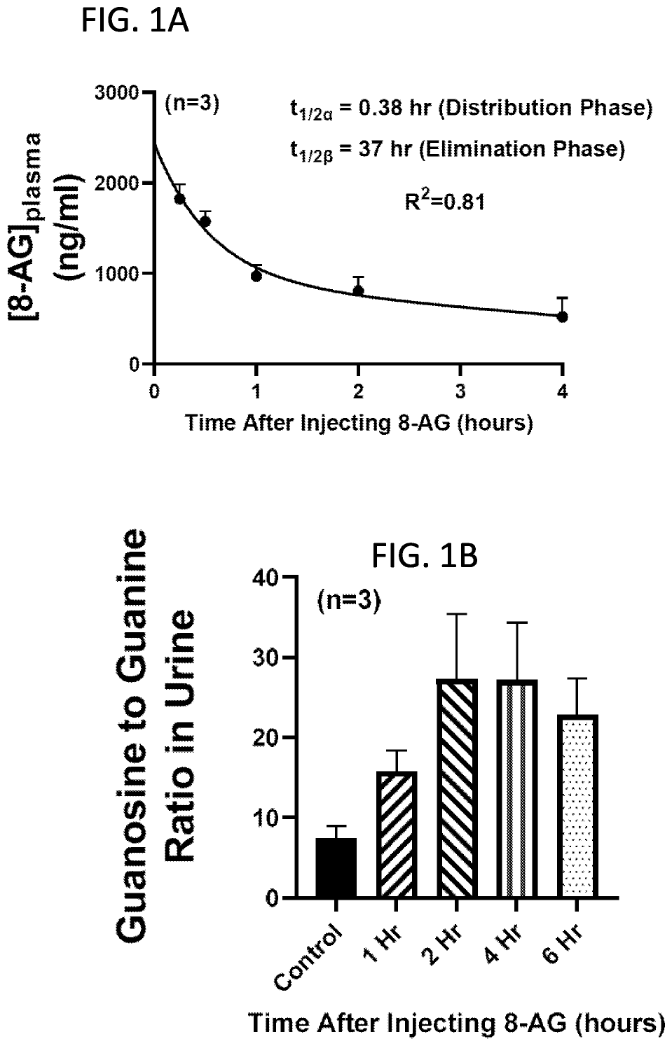

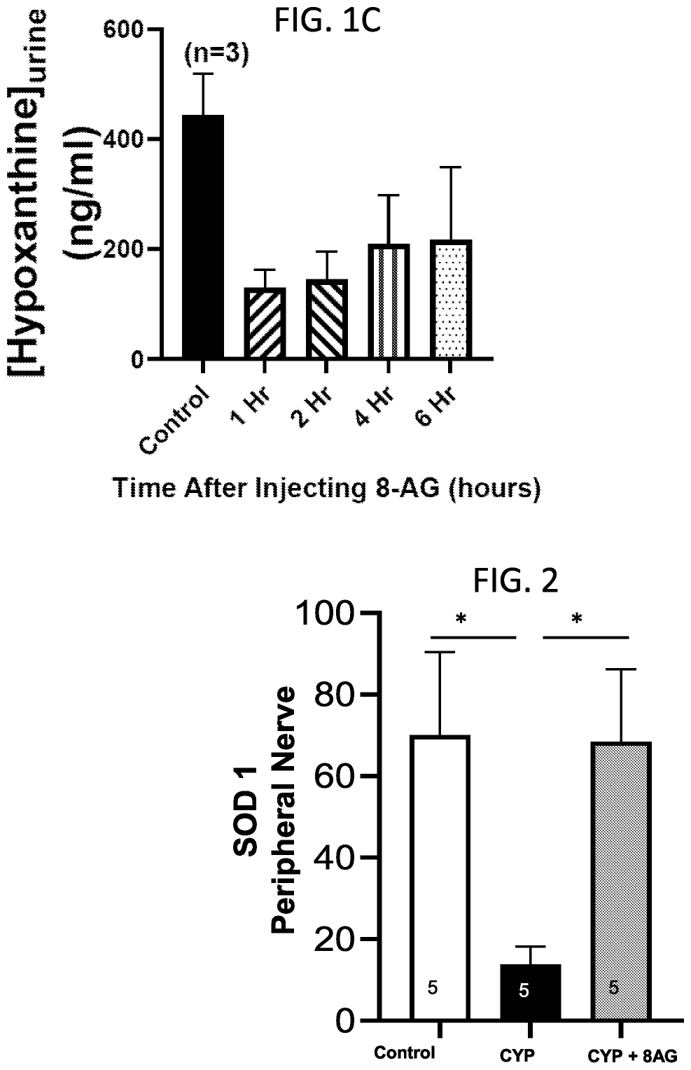

The plasma disposition of 8-AG showed a bi-exponential decay (FIG. 1A), which is typical of most drugs. The first phase of decay had a half-life of 0.38 hours, indicating that 8-AG rapidly distributed from the plasma compartment into its final volume of distribution. The second phase had a half-life of 37 hours, suggesting that 8-AG was slowly eliminated from the body. Consistent with inhibition of PNPase, 8-AG caused a sustained (>6 hours) reduction in the ratio of urine guanosine to urine guanine (FIG. 1B) and a sustained (>6 hours) reduction in urine levels of hypoxanthine (FIG. 1C). The long terminal half-life of 8-AG and the sustained (>6 hours) inhibition of PNPase following a bolus dose indicates that 8-AG can be administered systemically using a convenient dosing regimen.

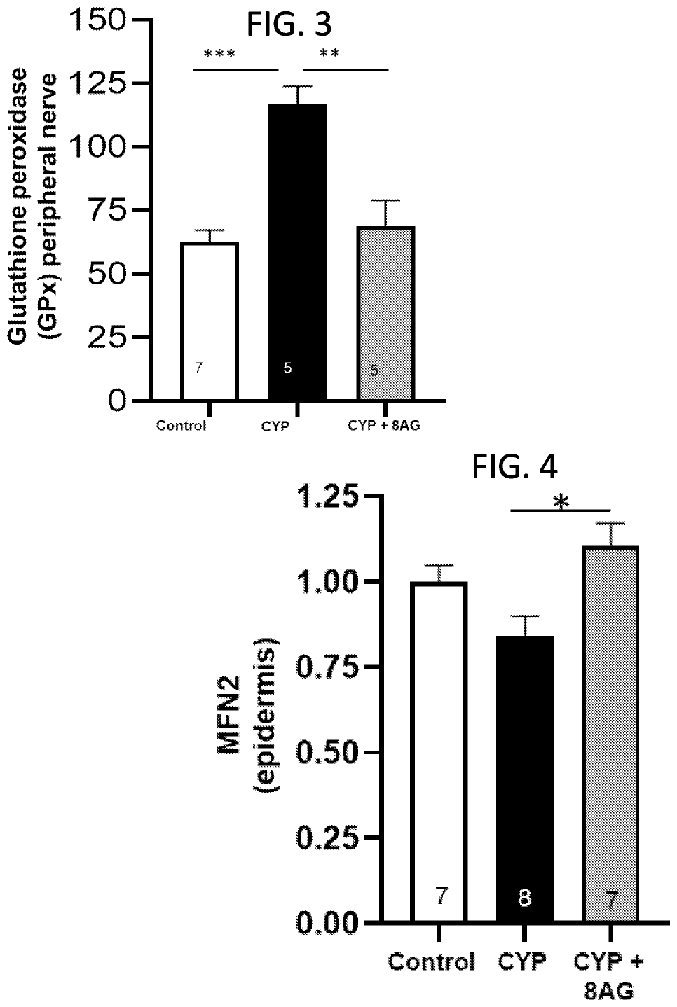

Administration of cyclophosphamide (CYP; a chemotoxic drug) reduced superoxide dismutase (SOD1) levels (FIG. 2) and increased glutathione peroxidase (GPx) activity (FIG. 3) in peripheral (sciatic) nerves. 8-AG prevented CYP-induced alterations in both SOD1 and GPx in rat peripheral nerves. SOD1 is an enzyme that breaks down potentially harmful oxygen molecules and thus is considered an important antioxidant defense in nearly all cells. The significant reduction of SOD1 by CYP in rat peripheral (sciatic) nerves may trigger excessive levels of tissue damaging hydroxyl radicals thereby leading to nerve damage. GPx activity is a crucial defense system to protect the nervous system against injury from augmented free radicals; likely the increase in GPs is a compensatory response to the reduction of SOD1. The prevention of CYP-induced alterations of SOD1 and GPx by 8-AG suggests that 8-AG protects against neuropathy induced by chemotoxic drugs.

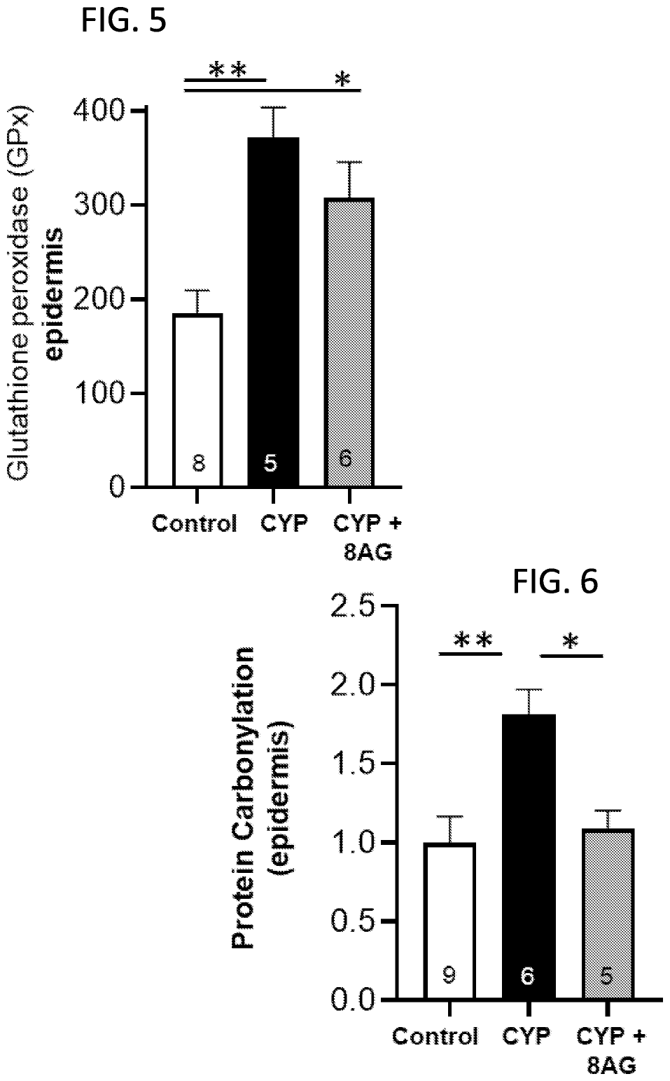

As indicated in FIG. 4, CYP resulted in a trend toward decreased levels of mitofusin 2 (MFN2) in the rat hindpaw footpad (epidermis), a biomarker which has been linked with impaired mitochondrial function. The decreased expression was reversed with 8-AG treatment. Also in the rat epidermis, it was observed that CYP significantly elevated both glutathione peroxidase activity (GPx; FIG. 5) as well as protein carbonylation (FIG. 6). The latter is a post-translational modification induced by excessive reactive oxygen species (ROS) and used as a marker for oxidative stress. For CYP-induced changes in both GPx and protein carbonylation, 8-AG treatment restored values to that of a control state.

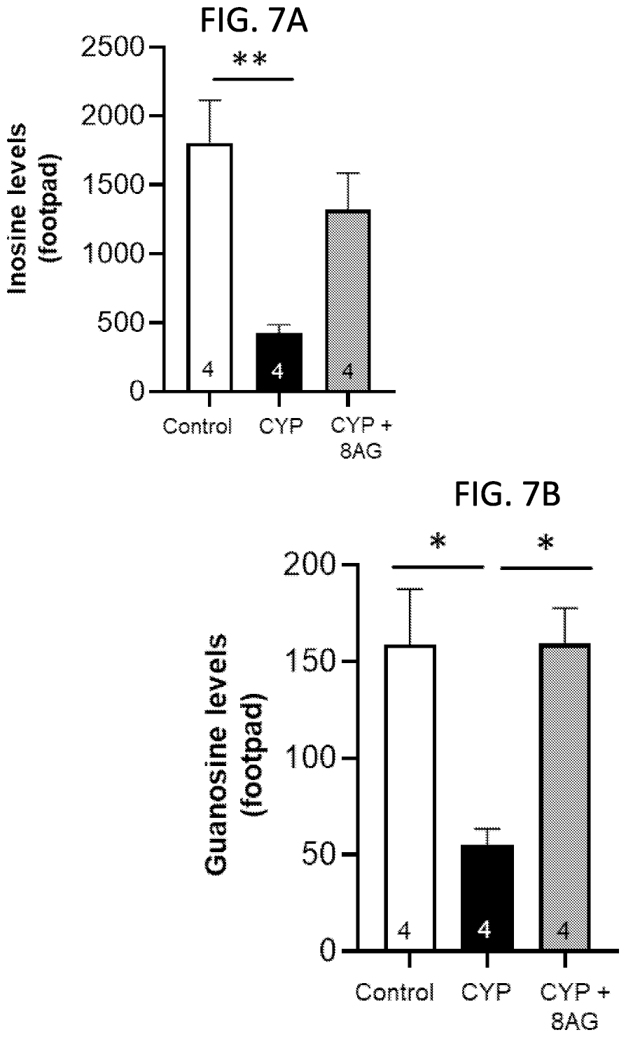

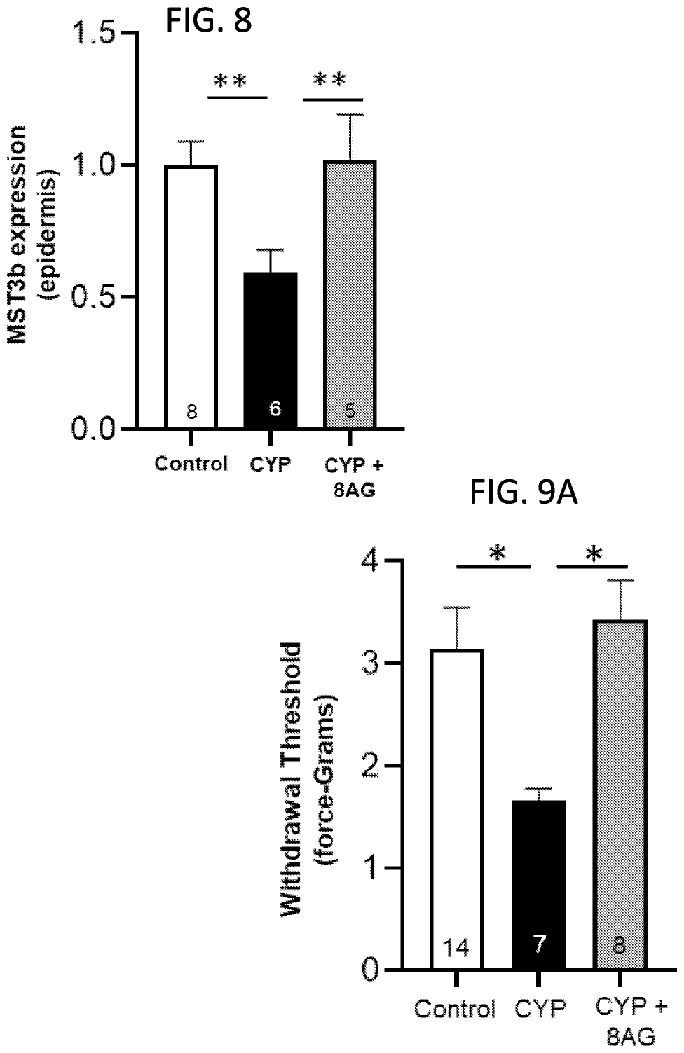

In the rat epidermis, CYP treatment significantly lowered levels of the tissue protective purines, inosine (FIG. 7A) and guanosine (FIG. 7B), and treatment with 8-AG completely restored tissue levels of both inosine (FIG. 7A) and guanosine (FIG. 7B). Further, in the rat epidermis CYP—CIPN significantly reduced expression of the enzyme Mst3b (FIG. 8), and this effect was completely prevented by 8-AG. Guanosine exhibits a number of protective effects against apoptosis and stimulates nerve regeneration; while inosine produces anti-inflammatory effects and can accelerate nerve regeneration after injury. Mst3b is a purine sensitive Ste20-like protein kinase that has a role in regeneration of both central and peripheral nerve fibers.

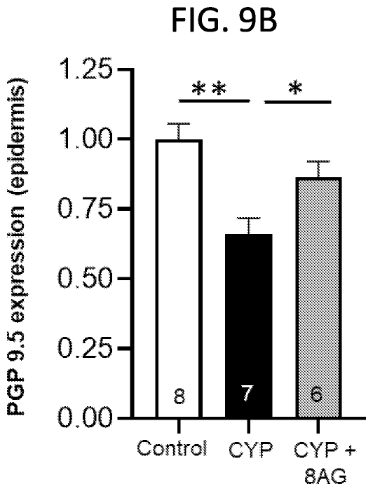

CYP was associated with significant increases in rat epidermal sensitivity to mechanical pressure (reduction in the minimum pressure in grams needed to elicit a withdrawal response) (FIG. 9A). As shown in FIG. 9B, CYP-reduced total PGP 9.5 epidermal expression, suggesting a change in nerve density and/or nerve damage. Both outcome measures were restored to control values with 8-AG treatment.

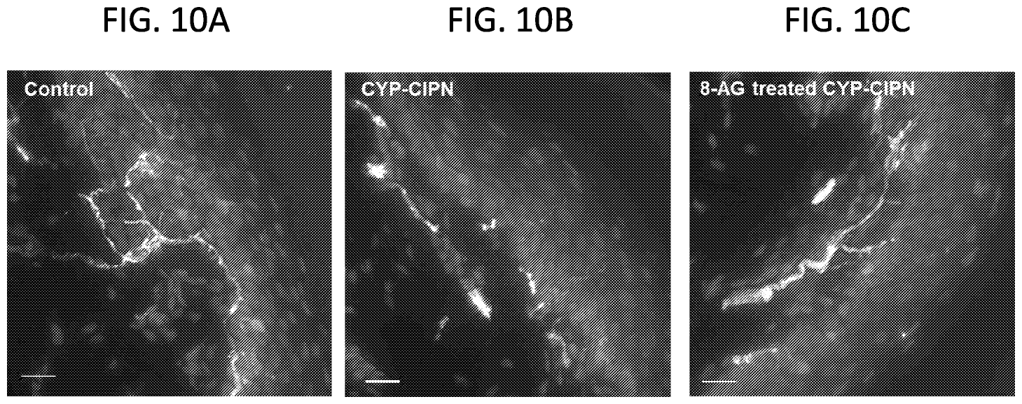

FIGS. 10A-8C illustrate the effects of oral 8-AG treatment on the distribution of the neuronal marker PGP 9.5 in rat epidermis in CYP—CIPN. Shown are representative fluorescent images of the pan-neuronal marker PGP 9.5 in Control (FIG. 10A), CYP—CIPN (FIG. 10B) and 8-AG-treated CYP—CIPN (FIG. 10C) rat epidermis. As shown in FIG. 10B that CYP—CIPN resulted in a ragged/damaged appearance of fine nerve terminals (which often do not reach to the epithelial surface). This was accompanied by gross edema (FIG. 10B, yellow arrow). Both neural damage and edema were normalized with 8-AG treatment (FIG. 10C).

The respiratory control ratio (RCR), a measure of the “tightness of coupling” between electron transport and oxidative phosphorylation, was determined from the ratio of state 3 to state 4 rates of respiration. As compared to the Control, CYP resulted in a decrease in RCR which was reversed to values similar to Control with 8-AG treatment. Studies have shown that the RCR significantly decreases with injury severity. Neuronal survival is linked to mitochondrial homeostasis.

CIPN is the most common side effect caused by antineoplastic treatment. The severity of CIPN dramatically affects patients' quality of life and can persists for many years (even after chemotherapy is withdrawn). Currently there are no established agents that can be recommended for the prevention of CIPN in patients with cancer undergoing treatment with potentially neurotoxic agents. The present study shows that in CIPN, 8-AG, a potent inhibitor of purine nucleoside phosphorylase (PNPase), reduces pain (as assessed by behavioral responses to adverse sensory stimuli), normalizes biomarkers for cellular injury, attenuates mitochondrial dysfunction, decreases oxidative stress and recovers the loss/degeneration of peripheral nerve fibers.

As demonstrated herein, for the treatment of CIPN, oral treatment with 8-AG is effective in restoring the expression of a key regulator of axonal growth, Mst3b; moreover, 8-AG normalizes tactile (mechanical) sensitivity in CIPN. The fact that Mst3b is sensitive to the purine nucleoside inosine supports the mechanism of 8-AG induced protection is inhibition of PNPase. Without being bound by theory, because PNPase inhibition blocks the metabolism of inosine to hypoxanthine and guanosine to guanine, the neuro-protective effects of PNPase inhibitors in general, and 8-AG in particular, can be mediated by increases in tissue levels of inosine and guanosine (both are neuro-protective purines which play a role in neural regeneration after injury) and reductions in tissue levels of hypoxanthine (damaging purine).

### Example 3

### Additional Studies

### Use of Taxanes (Paclitaxel) in an Animal Model for CIPN-Supplemental Data

A comparison of cyclophosphamide (Cytoxan) to the effects of paclitaxel (dose 2 mg/kg i.p.) alone versus control (vehicle) and 8-aminoguanine treated paclitaxel in adult Sprague-Dawley rats is performed, using the outcome measure of behavioral responses to mechanical stimuli (von Frey sensory testing) as well as protein carbonylation (a measure of oxidative stress) and the macrophage marker CD68.

### Spinal Cord Glial Cells and Neuropathic Pain

The expression levels of spinal cord glial cells (microglia; astrocytes), which are known to play a critical role in pain sensitization after nerve injury, are evaluated. These cells drive the neuroinflammatory signaling in the spinal cord that is characteristic of CIPN and other neuropathies. Microglia highly express the enzyme PNPase. Thus, microglial activation is evaluated in the models of CIPN and the effectiveness of 8-aminoguanine is also evaluated. Microglia expression changes with CYP and is reversed with 8-aminoguanine.

### Example 4

### Additional Materials and Methods

Materials: 8-Aminoguanine (8-AG) was purchased from Toronto Research Chemicals (Toronto, Ontario, Canada).

Animals: This study employed female Harlan Sprague Dawley (HSD) rats (3-4 mo; Envigo, Indianapolis, IN) and were divided into the following groups: 1) Control, 2) Cyclophosphamide (CYP, 75 mg/kg i.p. in saline vehicle; administered on days 0, 3 and 6 until sacrifice on day 8); 4) Paclitaxel (2 mg/kg i.p., 2 mg/kg i.p. in saline from a stock solution of 50/50 CremophorEL/ethanol); administered on days 0, 2, 4 and 6 until sacrifice on day 8; 4) 8-aminoguanine (8-AG; 5 mg/kg/day in drinking water; beginning 2 weeks prior to either CYP or PAC and continuing until sacrifice (day 8)). The dose of 8-AG was based on the experiments described above. In addition, the water consumption/day was tracked, and it was determined there was no variability (demonstrating consistency) in terms of dosing. All tissues (spinal cord and hindpaw epidermis) were removed from isoflurane anesthetized rats and the tissue was used for various experimental approaches as described below. At the end of tissue removal, animals were sacrificed by thoracotomy.

### Protocol 1: Behavioral Testing, Biomarkers and Mitochondrial Respiration

Von Frey sensory testing, Control, PAC and 8-AG-treated PAC rats were placed in an elevated chamber on a wire mesh floor and after acclimatization, calibrated von Frey microfilaments were applied to determine paw withdrawal threshold (PWT, in grams). Briefly, microfilaments (Stoelting Corp, USA) were applied with different forces perpendicularly to the plantar surface of both ipsilateral and contralateral hindpaw by increasing and decreasing stimulus intensity. The mechanical withdrawal threshold was the minimum pressure exerted (in grams) that triggered the paw withdrawal response (using the ‘up-down’ method). All animals were tested multiple times during the experimental paradigm and averages of the individual paw withdrawal response were calculated to find the rats 50% withdrawal threshold with the development of mechano-allodynia will be taken as a significant reduction in PWT (g) that failed to elicit withdrawal responses before chemotherapy treatment.

Western immunoblotting: Following the testing paradigms, rats were deeply anesthetized and tissues (e.g., epidermis and spinal cord segments (L3-L5) and a portion of each preparation was homogenized using Lysing Matrix D in a FastPrep 24 instrument (MP Biomedicals, Solon, OH) in HBSS [5 mM KCl, 0.3 mM KH₂PO₄, 138 mM NaCl, 4 mM NaHO₃, 0.3 mM Na₂HCO₃, 0.3 mM Na₂HPO₄, 5.6 mM glucose and 10 mM Hepes, pH 7.4 containing complete protease inhibitor cocktail (1 tablet/10 ml, Roche, Indianapolis, IN)] and phosphatase inhibitor cocktail (Sigma, 1:100). After centrifugation (13000 rpm; 15 min at 4° C.), the membrane protein fraction was prepared by suspending the membrane pellets in lysis buffer containing 0.3 M NaCl, 50 mM Tris-HCl (pH 7.6) and 0.5% Triton X-100 and the same concentration of protease inhibitors as above. The suspensions were incubated on ice and centrifuged (13000 rpm: 15 min at 4° C.). The protein concentrations of the supernatants were determined using the Pierce BCA protein assay (Thermo Scientific, Rockford, IL). After denaturation (100° C. for 5 min) in the presence of Laemmli sample buffer, lysate from each sample was separated on a 4-15% TGX Stain-Free SDS-PAGE gel (Bio-Rad Laboratories, Hercules, CA). As a loading control, total protein per sample was determined using Bio-Rad Stain Free SDS-PAGE gel technology. UV-activated protein fluorescence was imaged on a ChemiDoc MP (Bio-Rad). After proteins were transferred to polyvinylidene fluoride membranes, non-specific binding was blocked with 5% (w/v) non-fat dried milk dissolved in TBS-T (20 mM Trizma, 137 mM NaCl, 0.1% Tween-20, pH 7.6) for one hour, rinsed with TBS-T, and incubated overnight at 4° C. with primary antibody diluted in TBS-T containing 5% (w/v) milk or 5% BSA. After washing in TBS-T, the membranes were incubated with secondary antibody [sheep anti-mouse HRP (1:5000; GE Amersham, Pittsburgh, PA) or donkey anti-rabbit HRP (1:10,000; Advansta, San Jose, CA] for 1 hour in 5% (w/v) Milk TBS-T, washed, and incubated in WesternBright Quantum (Advansta, San Jose, CA) and then imaged on a ChemiDoc MP (BioRad). The volume (intensity) of each protein species was determined and normalized to total protein using Image Lab software (Bio-Rad). Primary antibody used was CD68 (Abcam recombinant anti-CD68 antibody; ab283654; 1:2000, 5% BSA TBS-T) and protein carbonylation was performed with an antibody that detects dinitrophenylhydrazine (DNPH)-derivatized carbonyl groups, following kit instructions (OxyBlot Protein Oxidation Detection Kit, S7150, Millipore, Burlington, MA).

Immunocytochemistry; A portion of tissues (a 3 mm biopsy from the rat hindpaw food pad or epidermis) was placed overnight in fixative (4% PFA) and cryoprotected (30% sucrose-phosphate buffered saline). Fluorescence immunocytochemistry was used to process cross-sections (20 μm) using an antibody (validated in rat tissue) against PGP 9.5 (Invitrogen PA5-29012, neuron specific protein; 1:500 dilution). After overnight incubation with the primary antibody, sections were incubated with secondary antibody (donkey anti-rabbit Alexa Fluor 555, Invitrogen A-31572, 1:1000) for one hour, washed and nuclei were counterstained with DAPI (4′, 6-diamidino-2-phenylindole, Invitrogen D21490, 1:2500, 10 minutes), washed and cover slipped with ProLong Gold (Invitrogen P36934). Representative images were taken and within each section 3-5 fields (each of 550 μm×700 μm) were imaged per region of interest.

Mitochondrial Respiration. Homogenates (spinal cord) were used to measure mitochondrial respiration. Mitochondria were isolated as described earlier with modifications. Briefly, after deep anesthesia with isoflurane (5%), spinal cord segments L3-L5 (≈80 mg) were isolated by extrusion and placed in mitochondrial solution (5 mM HEPES, 125 mM KCl, 2 mM Pi, 20 μM EDTA, 5 mM MgCl₂ and 0.2 mg/ml BSA, pH 7.4). The tissue is then minced and homogenized then centrifuged (1,000 g, 10 min). Following a second centrifugation, the pellet (containing the mitochondria) was resuspended in 100 μl of mitochondrial solution, and 25-50 μl of the suspension placed in a gas-tight vessel containing a Clark-type oxygen microelectrode (MI-730/OM-4; Microelectrodes, Londonderry, NH) to measure the state 3 (succinate+ADP) and state 4 (succinate alone) respiratory rates. The electrode was calibrated considering a total amount of dissolved O₂ in aqueous solution after equilibration with air at 36° C. to be 215 μM, zeroed with sodium dithionite. The respiratory control ratio (RCR), a measure of the “tightness of coupling” between electron transport and oxidative phosphorylation, was determined from ratio of state 3 to state 4 rates of respiration. An RCR of 2-4 is considered acceptable for complex ii substrates.

Statistics: Data were analyzed in GraphPad Prism 6 (GraphPad, La Jolla, CA) using an ordinary one-way ANOVA followed by Tukey's multiple comparison tests. All statistical tests were ordinary one-way ANOVA followed by Tukey's multiple comparison tests. p<0.05 was considered significant. Results are expressed as means±SEM.

### Example 5

### Additional Results

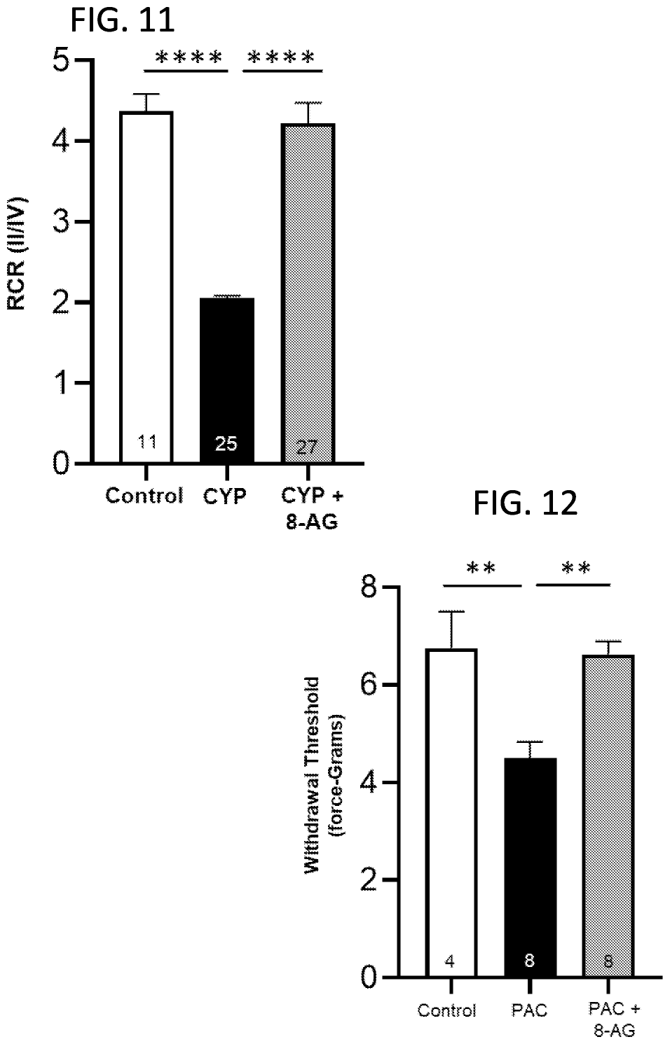

FIG. 11 shows a representative graph of mitochondria isolated from spinal cord (L3-L5) homogenates used to measure mitochondrial respiration. The respiratory control ratio (RCR), a measure of the “tightness of coupling” between electron transport and oxidative phosphorylation, was determined from the ratio of state 3 to state 4 rates of respiration. Compared to the Control, CYP resulted in a decrease in RCR which was reversed to values similar to Control with 8-AG treatment. Studies have shown that the RCR significantly decreases with injury severity. Neuronal survival is linked to mitochondrial homeostasis.

Administration of paclitaxel (PAC) was associated with significant increases in rat epidermal sensitivity to mechanical pressure (reduction in the minimum pressure in grams needed to elicit a withdrawal response) (FIG. 12). This outcome measure was restored to control values with 8-AG treatment.

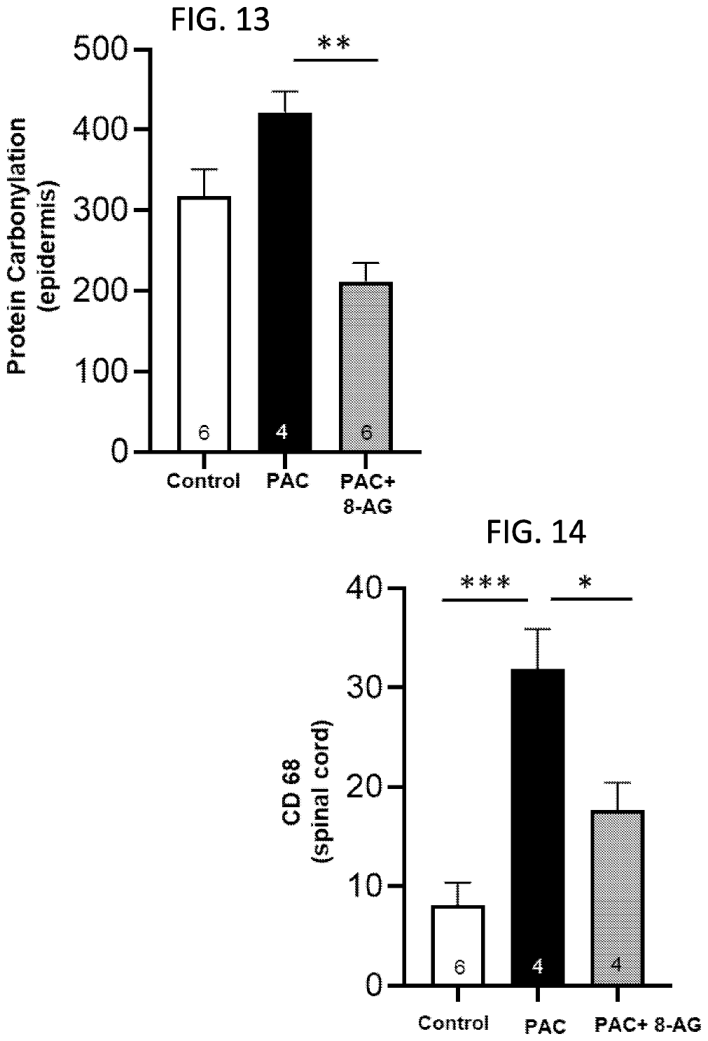

In the rat epidermis, it was observed that PAC significantly elevated protein carbonylation (FIG. 13), a post-translational modification induced by excessive reactive oxygen species (ROS) and used as a marker for oxidative stress. For PAC-induced changes in protein carbonylation, 8-AG treatment restored values to that of a control state.

Further, in the rat spinal cord, PAC-CIPN significantly increased expression of the macrophage biomarker CD68 (FIG. 14), and this effect was significantly reduced by 8-AG. Increased density of CD68 effector macrophages has been associated with neuropathic pain models and depletion of these pro-inflammatory macrophages has been shown to reduce sensitivity to neuropathic pain.

These data provide insight into the pathophysiology of many types of peripheral neuropathies including drug-induced, injury-induced or idiopathic neuropathies. CIPN is the most common side effect caused by antineoplastic treatment. The severity of CIPN dramatically affects patients' quality of life and can persists for many years (even after chemotherapy is withdrawn). The present study shows that in CIPN, 8-AG, a potent inhibitor of purine nucleoside phosphorylase (PNPase), reduces pain (as assessed by behavioral responses to adverse sensory stimuli), normalizes biomarkers for cellular injury, attenuates mitochondrial dysfunction, decreases oxidative stress and recovers the loss/degeneration of peripheral nerve fibers.

### Example 6

### Further Materials and Methods

Materials. 8-Aminoguanine (8-AG) was purchased from Toronto Research Chemicals (Toronto, Ontario, Canada).

Animals. This study employed female Harlan Sprague Dawley (HSD) rats (3-4 mo; Envigo, Indianapolis, IN) and were divided into the following groups: 1) Control, 2) Sciatic nerve crush (day 0, sacrifice on day 6); 3) 8-aminoguanine (8-AG; 5 mg/kg/day in drinking water; beginning 2 weeks prior to nerve crush and continuing until sacrifice (day 6)). The dose of 8-AG is based on the work above and published experiments. Further, the water consumption/day was tracked. There was no variability (demonstrating consistency) in terms of dosing. All tissues (hindpaw epidermis) were removed from isoflurane anesthetized rats and the tissue was used for various experimental approaches as described below. At the end of tissue removal, animals were sacrificed by thoracotomy.

### Protocol 1: Behavioral Testing and Biomarkers

Von Frey sensory testing. Control, sciatic nerve crush (one of the most common models of peripheral nerve injury in rodents) and 8-AG-treated sciatic nerve-crush rats were placed in an elevated chamber on a wire mesh floor and after acclimatization, calibrated von Frey microfilaments were applied to determine paw withdrawal threshold (PWT, in grams). Briefly, microfilaments (Stoelting Corp, USA) were applied with different forces perpendicularly to the plantar surface of both ipsilateral and contralateral hindpaw by increasing and decreasing stimulus intensity. The mechanical withdrawal threshold was the minimum pressure exerted (in grams) that triggered the paw withdrawal response (using the ‘up-down’ method). All animals were tested multiple times during the experimental paradigm and averages of the individual paw withdrawal response were calculated to find the rats 50% withdrawal threshold with the development of mechano-allodynia will be taken as a significant reduction in PWT (g) that failed to elicit withdrawal responses before sciatic nerve injury.

Western immunoblotting: Following the testing paradigms, rats were deeply anesthetized and tissues (e.g., epidermis was homogenized using Lysing Matrix D in a FastPrep 24 instrument (MP Biomedicals, Solon, OH) in HBSS [5 mM KCl, 0.3 mM KH₂PO₄, 138 mM NaCl, 4 mM NaHO₃, 0.3 mM Na₂HCO₃, 0.3 mM Na₂HPO₄, 5.6 mM glucose and 10 mM Hepes, pH 7.4 containing complete protease inhibitor cocktail (1 tablet/10 ml, Roche, Indianapolis, IN)] and phosphatase inhibitor cocktail (Sigma, 1:100). After centrifugation (13000 rpm; 15 min at 4° C.), the membrane protein fraction was prepared by suspending the membrane pellets in lysis buffer containing 0.3 M NaCl, 50 mM Tris-HCl (pH 7.6) and 0.5% Triton X-100 and the same concentration of protease inhibitors as above. The suspensions were incubated on ice and centrifuged (13000 rpm: 15 min at 4° C.). The protein concentrations of the supernatants were determined using the Pierce BCA protein assay (Thermo Scientific, Rockford, IL). After denaturation (100° C. for 5 min) in the presence of Laemmli sample buffer, lysate from each sample was separated on a 4-15% TGX Stain-Free SDS-PAGE gel (Bio-Rad Laboratories, Hercules, CA). As a loading control, total protein per sample was determined using Bio-Rad Stain Free SDS-PAGE gel technology. UV-activated protein fluorescence was imaged on a ChemiDoc MP (Bio-Rad). After proteins were transferred to polyvinylidene fluoride membranes, non-specific binding was blocked with 5% (w/v) non-fat dried milk dissolved in TBS-T (20 mM Trizma, 137 mM NaCl, 0.1% Tween-20, pH 7.6) for one hour, rinsed with TBS-T, and incubated overnight at 4° C. with primary antibody diluted in TBS-T containing 5% (w/v) milk or 5% BSA. After washing in TBS-T, the membranes were incubated with secondary antibody [sheep anti-mouse HRP (1:5000; GE Amersham, Pittsburgh, PA) or donkey anti-rabbit HRP (1:10,000; Advansta, San Jose, CA] for 1 hour in 5% (w/v) Milk TBS-T, washed, and incubated in WesternBright Quantum (Advansta, San Jose, CA) and then imaged on a ChemiDoc MP (BioRad). The volume (intensity) of each protein species was determined and normalized to total protein using Image Lab software (Bio-Rad). Primary antibody used was SOD1 (Cell Signaling 37385S, 1:15,000, 5% BSA TBS-T).

Statistics: Data were analyzed in GraphPad Prism 6 (GraphPad, La Jolla, CA) using an ordinary one-way ANOVA followed by Tukey's multiple comparison tests. All statistical tests were ordinary one-way ANOVA followed by Tukey's multiple comparison tests. p<0.05 was considered significant. Results are expressed as means±SEM.

### Example 7

### Spinal Cord Glial Cells and Neuropathic Pain

Spinal cord glial cells (microglia) are known to play a critical role in pain sensitization after nerve injury. These cells drive the neuroinflammatory signaling in the spinal cord that is characteristic of CIPN and other neuropathies.

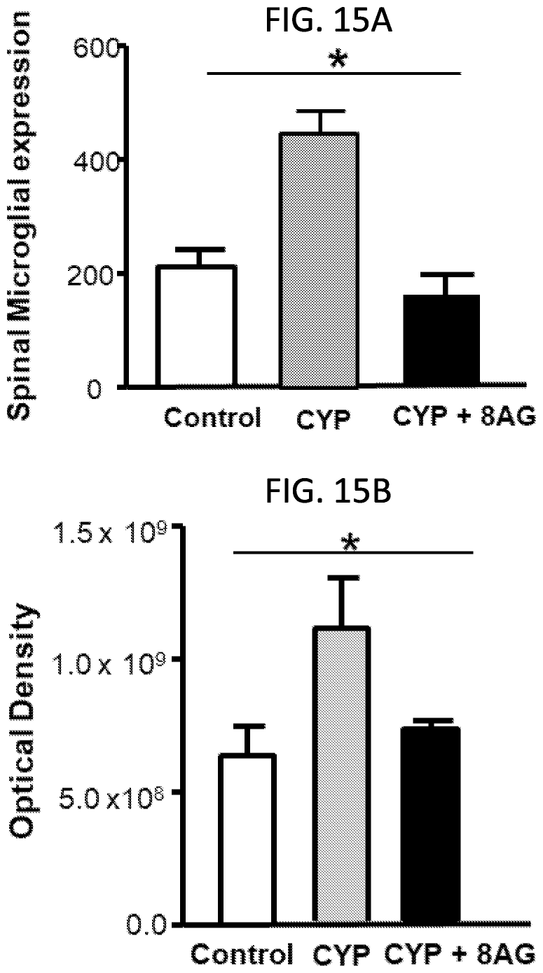

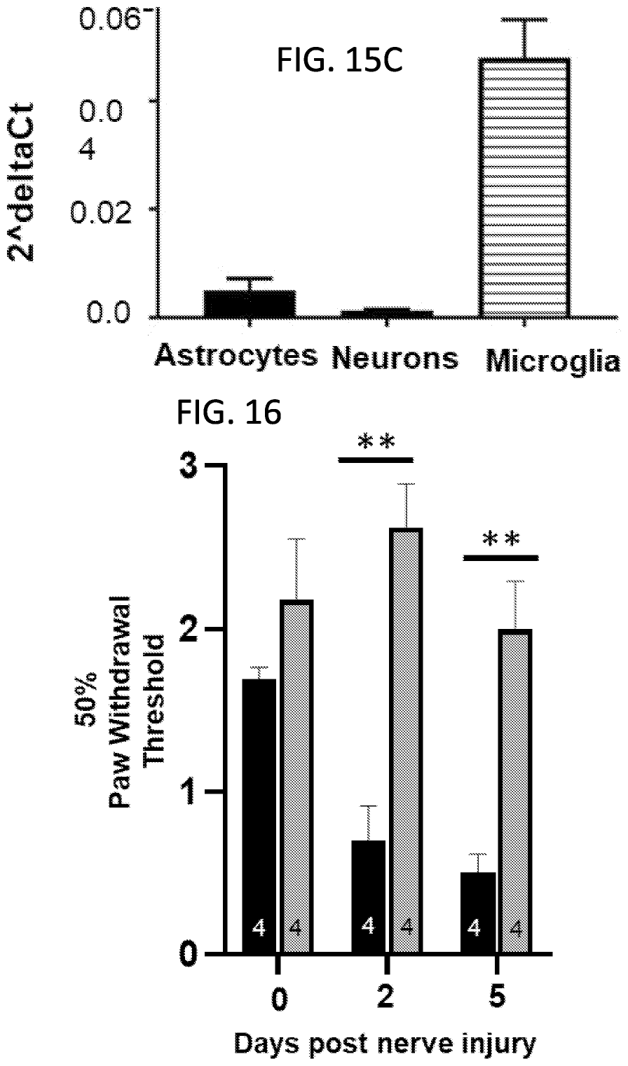

As shown in FIG. 15C, microglia highly express the enzyme PNPase (as compared to either astrocytes or neurons). Thus, the effectiveness of 8-aminoguanine in CYP—CIPN microglial expression and activation was evaluated. The calcium binding protein IBA-1 is expressed in microglia and is upregulated during the activation of these cells following nerve injury, ischemia and several other diseases. CYP—CIPN significantly increases IBA-1 expression (FIG. 15A, using Western immunoblotting) and increases spinal IBA-1 activation (FIG. 15B, measured by densitometry).

Microglia are able to increase secretion of proinflammatory mediators which contributes to ongoing nociception. These changes were normalized with 8-aminoguanine treatment.

### Example 8

### Effect of 8-Aminoguanine Treatment on Neural Function in Rodents Treated Following a Peripheral Nerve Injury

Peripheral nerve (sciatic nerve) injury was associated with significant increases in rat epidermal sensitivity to mechanical pressure (reduction in the minimum pressure in grams needed to elicit a withdrawal response) which was measured at day 2 and day 5 post injury (FIG. 16). These outcome measures were restored to control values with 8-AG treatment.

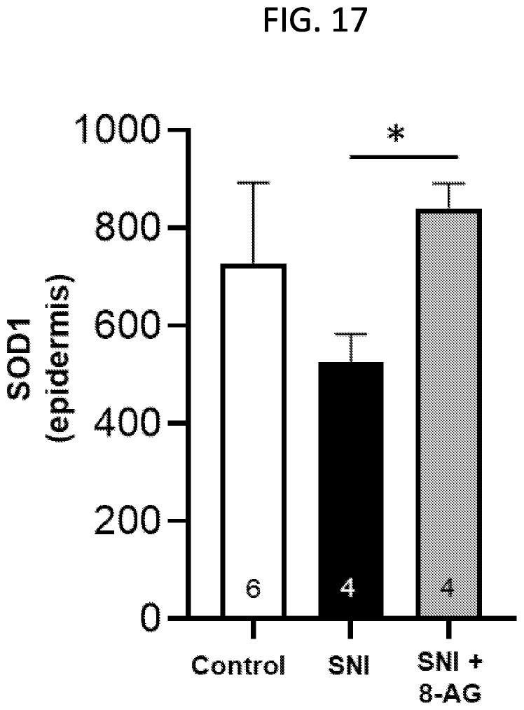

FIG. 17 illustrates the effects of oral 8-AG treatment on the expression of superoxide dismutase (SOD1) in rat epidermis following sciatic nerve injury (SNI). SOD1 is an enzyme that breaks down potentially harmful oxygen molecules and thus is considered an important antioxidant defense in nearly all cells. The reduction of SOD1 by nerve damage in rat epidermis may trigger excessive levels of tissue damaging hydroxyl radicals thereby leading to further damage. This outcome measure was restored to control values by 8-AG treatment.

Peripheral neuropathy describes a collection of disorders marked by damage to the peripheral nerves and can be caused by a range of underlying conditions (e.g., diabetes, physical injuries, vascular problems, infection, exposure to toxic chemicals). Several animal models of peripheral nerve injury exist including sciatic nerve crush which develop behaviors of hypersensitivity, one of the hallmark signs of neuropathic pain in humans. The severity of symptoms dramatically affects patients' quality of life and can persist beyond the time of normal tissue healing and management of this type of pain is challenging with treatment requiring a multidisciplinary approach. The present study showed that in sciatic nerve damage, 8-AG, a potent inhibitor of purine nucleoside phosphorylase (PNPase), reduced pain (as assessed by behavioral responses to adverse sensory stimuli) and decreases oxidative stress.

8-Aminoguanine normalized epidermis injury-induced decreases in rat epidermal superoxide dismutase (SOD1), used as a marker of increased oxidative stress. 8-Aminoguanine also normalized injury-induced increases in tactile (mechanical) sensitivity. This evidences that PNPase inhibitors, such as 8-aminoguanine, are a class of agents that reverse injury-induced peripheral neuropathy by: 1) improving sensory function; 2) reducing oxidative stress. PNPase inhibitors are effective treatments for peripheral neuropathy. The evidence further supports systemic administration of PNPase inhibitors, such as 8-aminoguanine, for the treatment of peripheral neuropathy.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of treating a subject with a peripheral neuropathy, comprising:
selecting the subject with the peripheral neuropathy; and
administering to the subject a therapeutically effective amount of a purine nucleoside phosphorylase (PNPase) inhibitor and/or a PNPase purine nucleoside substrate, thereby treating the peripheral neuropathy in the subject.

2. A method of improving neuron function of a subject with peripheral neuropathy, comprising:
selecting the subject with the peripheral neuropathy, wherein the subject is in need of improving function of peripheral nerves, and
administering to the subject an amount of a purine nucleoside phosphorylase (PNPase) inhibitor and/or a PNPase purine nucleoside substrate effective to improve function of peripheral neurons in the subject,
thereby improving neuron function in the subject.

3. The method of claim 1, wherein the method improves sensory nerve function of the subject.

4. The method of claim 1, wherein the method improves motor neuron function in the subject.

5. The method of claim 1, wherein the PNPase inhibitor is a guanine comprising a substituent at the 8-position, a guanosine comprising a substituent at the 8-position, an inosine comprising a substituent at the 8-position, a hypoxanthine comprising a substituent at the 8-position, a PNPase transition state analog, or a pharmaceutically acceptable salt thereof.

6. The method of claim 5, wherein the substituent is amine, hydroxyl, nitro, nitroso, alkoxy, carbonyl, halogen, carboxyl, ester, carbonate, amide, or haloaliphatic.

7. The method of claim 5, wherein the substituent is amine.

8. The method of claim 5, wherein the guanine comprising a substituent at the 8-position is 8-aminoguanine.

9. The method of claim 5, wherein the PNPase transition state analog is:
7-[(2S,3S,4R,5R)-3,4-dihydroxy-5-(hydroxymethyl)pyrrolidin-2-yl]-3H,4H,5H-pyrrolo[3,2-d]pyrimidin-4-one;
7-(((3R,4R)-3-hydroxy-4-(hydroxymethyl)pyrrolidin-1-yl)methyl)-3H-pyrrolo[3,2-d]pyrimidin-4(5H)-one;
7-(((2R,3S)-1,3,4-trihydroxybutan-2-ylamino)methyl)-3H-pyrrolo[3,2-d]pyrimidin-4(5H)-one;
7-((1,3-dihydroxypropan-2-ylamino)methyl)-3H-pyrrolo[3,2-d]pyrimidin-4(5H)-one; or
a pharmaceutically acceptable salt thereof.

10. The method of claim 5, wherein the pharmaceutically acceptable salt is a chloride salt.

11. The method of claim 1, wherein the PNPase inhibitor and/or a PNPase purine nucleoside substrate is administered systemically to the subject.

12. The method of claim 11, the PNPase inhibitor and/or a PNPase purine nucleoside substrate is administered orally, intravenously, or intramuscularly to the subject.

13. The method of claim 11, wherein administering comprises repeated delivering to the subject.

14. The method of claim 1, wherein the PNPase inhibitor is the guanine comprising a substituent at the 8-position or the guanosine comprising a substituent at the 8-position.

15. The method of claim 1, wherein the subject is a veterinary subject.

16. The method of claim 1, wherein the subject is a human subject.

17. The method of claim 1, wherein the peripheral neuropathy is induced by a toxic agent.

18. The method of claim 17, wherein the toxic agent is a chemotherapeutic agent.

19. The method of claim 17, wherein the toxic agent is a platinum compound, a taxane, a vinca alkaloid, an anti-microtubule agent, a proteasome inhibitor, or thalidomide.

20. The method of claim 17, wherein the toxic agent is radiation.

21. The method of claim 1, wherein the peripheral neuropathy is genetically acquired.

22. The method of claim 1, wherein the peripheral neuropathy is diabetic neuropathy or Guillian-Barre syndrome.

23. The method of claim 1, wherein the a) peripheral neuropathy results from a systemic or infectious disease, or b) the peripheral neuropathy is idiopathic.

24. The method of claim 1, wherein the peripheral neuropathy results from a post-surgical complication.

25. The method of claim 1, wherein the subject has neuropathic postural orthostatic tachycardia syndrome (POTS) or complex regional pain syndrome (CRPS).

26. The method of claim 1, wherein the method treats neuropathic pain in the subject.
